(19) United States
(12) Patent Application Publication
(10) Pub. No.: US 2003/0236443 A1
(43) Pub. Date: Dec. 25, 2003

(54) Methods and apparatus for the identification and stabilization of vulnerable plaque

(76) Inventors: Eduardo Ignacio Cespedes, Folsom, CA (US); Kenneth J. Michlitsch, Livermore, CA (US)

Correspondence Address: Nicola A. Pisano, Esq., Suite 200, 11988 El Camino Real, San Diego, CA, 92130, US

(21) Appl. No.: 10/393,665

(22) Filed: Mar. 20, 2003

Related U.S. Application Data
(63) Continuation-in-part of application No. 10/127,052, filed on Apr. 19, 2002.
(63) Continuation-in-part of application No. 10/232,428, filed on Aug. 28, 2002.

(51) Int. Cl. A61F 2/00

(52) U.S. Cl. 600/29

(57) ABSTRACT

The present invention provides methods and apparatus for identifying and stabilizing vulnerable plaque via multi-functional catheters having both infrared detection and imaging capabilities. It is expected that correlating imaging and infrared data will facilitate improved identification of vulnerable plaque. Apparatus of the present invention may also be provided with optional stabilization elements for stabilizing vulnerable plaque, as well as optional embolic protection. Methods of using apparatus of the present invention are also provided.

### REFERENCE TO RELATED APPLICATIONS

The present application is a continuation-in-part of U.S. patent application Ser. No. 10/232,429, filed Aug. 28, 2002, which is hereby incorporated by reference in its entirety, which is a continuation-in-part of U.S. patent application Ser. No. 10/127,052, filed Apr. 19, 2002.

### FIELD OF THE INVENTION

The present invention relates to methods and apparatus for identifying and stabilizing vulnerable plaque, and for characterizing plaque. More particularly, the present invention relates to specialized catheters having both an imaging element and a thermographer for improved identification of vulnerable plaque. Apparatus of the present invention may in addition include an optional stabilization element for stabilizing the plaque.

### BACKGROUND OF THE INVENTION

Vulnerable plaque is commonly defined as plaque having a lipid pool with a thin fibrous cap, which is often infiltrated by macrophages. Vulnerable plaque lesions generally manifest only mild to moderate stenoses, as compared to the large stenoses associated with fibrous and calcified lesions. While the more severe stenoses of fibrous and calcified lesions may limit flow and result in ischemia, these larger plaques often remain stable for extended periods of time. In fact, rupture of vulnerable plaque is believed to be responsible for a majority of acute ischemic and occlusive events, including unstable angina, myocardial infarction, and sudden cardiac death.

The mechanism behind such events is believed to be thrombus formation upon rupture and release of the lipid pool contained within vulnerable plaque. Thrombus formation leads to plaque growth and triggers acute events. Plaque rupture may be the result of inflammation, or of lipid accumulation that increases fibrous cap stress. Clearly, prospective identification and stabilization of vulnerable plaque is key to effectively controlling and reducing acute ischemic and occlusive events.

A significant difficulty encountered while attempting to identify and stabilize vulnerable plaque is that standard angiography provides no indication of whether or not a given plaque is susceptible to rupture. Furthermore, since the degree of stenosis associated with vulnerable plaque is often low, in many cases vulnerable plaque may not even be visible using angiography.

A variety of techniques for identifying vulnerable plaque are being pursued. These include imaging techniques, for example, Intravascular Ultrasound (“IVUS”), Optical Coherence Tomography (“OCT”), and Magnetic Resonance Imaging (“MRI”). Two primary IVUS techniques have been developed. The first is commonly referred to as rotational IVUS, which uses an ultrasound transducer that is rotated to provide a circumferential image of a patient's vessel. The second technique is commonly referred to as phased-array IVUS, which uses an array of discrete ultrasound elements that each provide image data. The image data from each element is combined to form a circumferential image of the patient's vessel.

Rotational IVUS systems are marketed by Terumo Corporation of Tokyo, Japan, and the Boston Scientific Corporation of Natick, MA, and are described, for example, in U.S. Pat. No. 6,221,015 to Yock, which is incorporated herein by reference. Phased-array IVUS systems are marketed by JOMED Inc., of Rancho Cordova, Calif., and are described, for example, in U.S. Pat. No. 6,283,920 to Eberle et al., as well as U.S. Pat. No. 6,283,921 to Nix et al., both of which are incorporated herein by reference. Optical Coherence Tomography systems are developed by Lightlab Imaging, LLC., of Westford, Mass., and are described, for example, in U.S. Pat. No. 6,134,003 to Tearney et al., which is incorporated herein by reference. U.S. Pat. No. 5,699,801 to Atalar et al., which also is incorporated herein by reference, describes methods and apparatus for Magnetic Resonance Imaging inside a patient's vessel.

A primary goal while characterizing plaque-type via an imaging modality is identification of sub-intimal lipid pools at the site of vulnerable plaque. In an IVUS study entitled, “Morphology of Vulnerable Coronary Plaque: Insights from Follow-Up of Patients Examined by Intravascular Ultrasound Before an Acute Coronary Syndrome” (Journal of the American College of Cardiology, 2000; 35:106-11), M. Yamagishi et al., concluded that, “the risk of rupture is high among eccentric lesions with a relatively large plaque burden and a shallow echolucent zone.” IVUS allows characterization of the concentricity or eccentricity of lesions, as well as identification of echolucent zones, which are indicative of lipid-rich cores. However, while IVUS and other advanced imaging modalities may provide a means for identifying vulnerable plaque and selecting patients likely to benefit from aggressive risk factor interventions, such imaging modalities typically require a significant degree of skill, training and intuition on the part of a medical practitioner in order to achieve a proper diagnosis.

In addition to imaging techniques, biological techniques also have been proposed for identifying vulnerable plaque. Biological techniques typically rely on characterization of material properties of the plaque. Biological techniques include thermography, biological markers, magnetic resonance, elastography and palpography. Biological markers typically attempt to ‘tag’ specific tissue types, for example, via chemical receptors, with markers that allow easy identification of tissue type. Magnetic resonance operates on the principal that different tissue types may resonate at different, identifiable frequencies. Techniques combining Magnetic Resonance Imaging and biological markers have also been proposed in which superparamagnetic iron oxide nanoparticles are used as MRI contrast media. It is expected that vulnerable plaque will preferentially take up the nanoparticles by virtue of macrophage infiltration, leaking vasa vasorum, and permeable thin cap (M. AbouQamar et al., Poster Abstract, Transcatheter Cardiovascular Therapeutics, 2001, Washington, D.C.).

Elastography and palpography seek to characterize the strain modulus, or other mechanical properties, of target tissue. Studies have shown that different plaque types exhibit different, identifiable strain moduli, which may be used to characterize plaque type. Elastography is described, for example, in U.S. Pat. No. 5,178,147 to Ophir et al., which is incorporated herein by reference. Palpography is described, for example, in U.S. Pat. No. 6,165,128 to Cespedes et al., which also is incorporated herein by reference.

Thermography seeks to characterize tissue type via tissue temperature. Tissue temperature may be characterized via thermographers of various types, including, for example, thermistors, thermosensors, thermocouples, thermometers, spectrography, spectroscopy, and infrared. Tissue characterization via thermographers has been known for some time; for example, U.S. Pat. No. 4,960,109 to Lele et al., which is incorporated herein by reference, describes a multi-function probe for use in hyperthermia therapy that employs at least one pair of temperature sensors.

It has been observed that vulnerable plaque results in a temperature increase at a vessel wall of as much as about 0.1° C. to over 2.0° C., and is typically at least 0.3° C. A review of thermographic apparatus and techniques for plaque characterization is provided by C. Stefanadis in “Plaque Thermal Heterogeneity—Diagnostic Tools and Management Implications” (Expert Presentation, Transcatheter Cardiovascular Therapeutics, Washington, D.C.). Thermography apparatus and methods are also provided in Greek Patent No. 1003158B to Diamantopoulos et al., Greek Patent No. 1003178B to Toutouzas et al., and Greek Utility Model No. 98200093U to Diamantopoulos et al., all of which are incorporated herein by reference. U.S. Pat. No. 5,445,157 to Adachi et al., which is incorporated herein by reference, describes a thermographic endoscope including an infrared image-forming device. U.S. Pat. No. 5,871,449 to Brown and U.S. Pat. No. 5,935,075 to Casscells et al., both incorporated herein by reference, describe catheters capable of detecting infrared radiation.

Although passing reference is made in the Abstract of the Casscells patent to using the infrared detection system with or without ultrasound, no ultrasound apparatus is described. If ultrasound were to be used, it would presumably be applied using known techniques, i.e. extravascularly or via a secondary, stand-alone IVUS catheter. Using extravascular ultrasound or a secondary, stand-alone IVUS catheter, in conjunction with an infrared catheter is expected to increase the complexity, time, and cost associated with identifying vulnerable plaque.

For the purposes of the present invention, in addition to temperature characterization, thermography includes characterization of tissue pH, for example, via Near-Infrared (“NIR”) Spectroscopy. T. Khan et al., have shown that inflamed regions of plaque exhibit lower pH, and that NIR Spectroscopy may be used to measure such pH (“Progress with the Calibration of A 3-French Near Infrared Spectroscopy Fiberoptic Catheter for Monitoring the pH Of Atherosclerotic Plaque: Introducing a Novel Approach For Detection of Vulnerable Plaque,” Poster Abstract, Transcatheter Cardiovascular Therapeutics, 2001, Washington, D.C.). Thus, plaque temperature and plaque pH are inversely correlated to one another. Thermography further may include other spectroscopic tissue characterization, such as tissue composition characterization.

Although thermography is a promising new technique for identifying vulnerable plaque, it has several drawbacks. First, since thermography doesn't provide image data, it is expected that medical practitioners will have difficulty determining proper locations at which to use a thermographer in order to characterize plaque type. Thus, secondary, stand-alone imaging apparatus may be required in order to adequately identify and characterize plaque. Requiring separate imaging and thermography apparatus is expected to increase complexity, time and cost associated with identifying vulnerable plaque. Additionally, thermography provides no indication of the eccentricity of a plaque or of the presence or magnitude of lipid pools disposed in the plaque, both of which have been shown to indicate the presence of vulnerable plaque.

U.S. Pat. No. 5,924,997 to Campbell and PCT Publication WO 01/74263 to Diamantopolous et al., both of which are incorporated herein by reference, describe or suggest vascular catheters providing ultrasound imaging and temperature detection. The Campbell reference contemplates thermography catheters having a lumen in which a standard ultrasonography catheter may be advanced. It is expected that the cross-sectional profile of such catheters would significantly limit their clinical applicability. Moreover, the catheters described in the Campbell patent do not appear to have any “window” for passage of the IVUS signals; thus, it is expected that such composite thermography/IVUS catheters would provide reduced bandwidth, fidelity, etc., as compared to stand-alone IVUS catheters. The Campbell reference also describes an integrated catheter having thermography and rotational IVUS, but does not clearly describe how such data could be correlated.

The device suggested in PCT Publication WO 01/74263 also has several drawbacks. That reference provides no enabling structure for coupling thermography data to IVUS images. Moreover, the PCT reference contemplates displaying imaging and thermography data in separate, positionally-linked windows, which is expected to increase difficulties in analyzing the data.

Both U.S. Pat. No. 5,924,997 and PCT Publication WO 01/74263 apparently do not acknowledge that patients may not have regions within their vasculature that are suspected of harboring vulnerable plaque. The added time, expense, etc., of using thermography in conjunction with IVUS or other imaging modalities may not be justified. Accordingly, it would be desirable to provide an imaging catheter through which separate thermography probes, e.g. functional measurement guide wires, optionally may be advanced, for example, only in patients suspected of harboring vulnerable plaque.

Another drawback associated with many of the prior art techniques for identifying and stabilizing vulnerable plaque is that identification and stabilization are typically achieved using separate apparatus. Stabilization techniques include both local and systemic therapy. Localized techniques include angioplasty, stenting, mild heating, photonic ablation, radiation, local drug injection, gene therapy, covered stents and coated stents, for example, drug-eluting stents. Systemic therapies include extreme lipid lowering; inhibition of cholesterol acyltransferase (Acyl-CoA, “ACAT”); matrix metalloproteinase (“MMP”) inhibition; and administration of statins, anti-inflammatory agents, anti-oxidants and/or Angiotensin-Converting Enzyme (“ACE”) inhibitors.

Multi-functional devices have been proposed in other areas of vascular intervention. For example, U.S. Pat. No. 5,906,580 to Kline-Schoder et al., which is incorporated herein by reference, describes an ultrasound transducer array that may transmit signals at multiple frequencies and may be used for both ultrasound imaging and ultrasound therapy. PharmaSonics, Inc., of Sunnyvale, Calif., markets therapeutic ultrasound catheters, which are described, for example, in U.S. Pat. No. 5,725,494 to Brisken et al., incorporated herein by reference. U.S. Pat. No. 5,581,144 to Corl et al., incorporated herein by reference, describes another ultrasound transducer array that is capable of operating at multiple frequencies.

In addition to multi-functional ultrasound devices, other multi-functional interventional devices are described in U.S. Pat. Nos. 5,571,086 and 5,855,563 to Kaplan et al., both of which are incorporated herein by reference. However, none of these devices, nor the multi-functional ultrasound devices discussed previously, are suited for rapid identification and stabilization of vulnerable plaque in accordance with the principles of the present invention.

In view of the drawbacks associated with previously known methods and apparatus for identifying and stabilizing vulnerable plaque, it would be desirable to provide methods and apparatus that overcome those drawbacks.

It would be desirable to provide methods and apparatus that reduce the skill and training required on the part of medical practitioners in order to identify and stabilize vulnerable plaque.

It would be desirable to provide methods and apparatus for identifying and stabilizing vulnerable plaque that reduce the cost, complexity and time associated with such procedures.

It would be desirable to provide methods and apparatus that are multi-functional.

It would be desirable to provide methods and apparatus that facilitate characterization of lesion eccentricity, echogenicity, temperature or pH, and tissue composition.

It would be desirable to provide methods and apparatus that combine imaging, thermography, infrared spectroscopy, biochemical sensing and/or optional vulnerable plaque stabilization elements in a single device.

It would be desirable to provide a variety of data characterization techniques.

It would be desirable to provide methods and apparatus for identifying and stabilizing vulnerable plaque that facilitate imaging and allow subsequent advancement of thermography apparatus through the imaging apparatus for detailed inspection of regions suspected of harboring vulnerable plaque.

### SUMMARY OF THE INVENTION

In view of the foregoing, it is an object of the present invention to provide apparatus and methods for identifying and stabilizing vulnerable plaque that overcome drawbacks associated with previously known apparatus and methods.

It is an object to provide methods and apparatus that reduce the skill and training required on the part of medical practitioners in order to identify and stabilize vulnerable plaque.

It also is an object to provide methods and apparatus for identifying and stabilizing vulnerable plaque that reduce the cost, complexity and time associated with such procedures.

It is another object to provide methods and apparatus that are multi-functional.

It is yet another object to provide methods and apparatus that facilitate characterization of lesion eccentricity, echogenicity, temperature or pH, and tissue composition.

It is an object to provide methods and apparatus that combine imaging, thermography, infrared spectroscopy, biochemical sensing and/or optional vulnerable plaque stabilization elements in a single device.

It would be desirable to provide a variety of data characterization techniques.

It is an object to provide methods and apparatus for identifying and stabilizing vulnerable plaque that facilitate imaging and allow subsequent advancement of thermography apparatus through the imaging apparatus for detailed inspection of regions suspected of harboring vulnerable plaque.

These and other objects of the present invention are accomplished by providing apparatus for identifying vulnerable plaque comprising a catheter having both an imaging element and a thermographer. Providing both thermography and imaging in a single, multi-functional catheter is expected to decrease the cost and increase the accuracy of vulnerable plaque identification, as well as simplify and expedite identification, as compared to providing separate, stand-alone thermography and imaging. Apparatus of the present invention also may be provided with optional stabilization elements for stabilizing vulnerable plaque, thereby providing vulnerable plaque identification and stablization in a single device.

In a first embodiment of the present invention, a catheter is provided having a phased-array IVUS imaging system and a plurality of thermocouples. The plurality of thermocouples may be deployed into contact with an interior wall of a patient's body lumen, thereby providing temperature measurements along the interior wall that may be compared to IVUS images obtained with the imaging system to facilitate identification of vulnerable plaque. In a second embodiment, a catheter is provided with a rotational IVUS imaging system and a fiber optic infrared thermography system. The infrared system's fiber optic is preferably coupled to the rotating drive cable of the rotational IVUS imaging system, thereby providing a full circumferential temperature profile along the interior wall of the patient's body lumen. In a third embodiment, a catheter is provided having a phased-array IVUS imaging system and a fiber optic infrared thermography system. The infrared system preferably comprises a plurality of fiber optics to provide a full circumferential temperature profile along the interior wall of a patient's body lumen.

In a fourth embodiment, apparatus of the present invention is provided with, in addition to an imaging element and a thermographer, an optional stabilization element. The apparatus may further comprise an optional embolic protection device to capture emboli and/or other material released, for example, during stabilization of vulnerable plaque. The stabilization element may comprise an inflatable balloon. In a fifth embodiment, the stabilization element comprises a second ultrasound transducer that resonates at therapeutic ultrasound frequencies, as opposed to ultrasonic imaging frequencies. As yet another embodiment, the imaging element of the present invention comprises an ultrasound transducer that is capable of transmitting multiple frequencies that are suited to both ultrasonic imaging and ultrasonic therapy, thereby providing both vulnerable plaque imaging and stabilization in a single element.

In a sixth embodiment, a catheter, preferably comprising an imaging transducer, is provided having a side exit port disposed on a lateral surface of the catheter, the side exit port defining a distal termination of a bifurcation of a single lumen or one of two lumens disposed within the catheter through which a thermographer, for example, a functional measurement guide wire, a fiber optic spectroscopy probe, or a fiber optic infrared probe, may be advanced. The catheter also may comprise a plurality of bifurcations or lumens through which a plurality of thermographers may be advanced to facilitate acquisition of a full circumferential temperature profile along the interior wall of a patient's body lumen. The distal portion of the above-mentioned lumens preferably comprise a curvature that directs advancement of the thermographer so that a distal working tip of the thermographer may be disposed in sensory proximity with the vessel wall to facilitate data acquisition.

Additionally, the direction provided by this curvature, along with the position of an optional imaging system disposed on the catheter distal the side exit port, e.g. an IVUS imaging system, permits the thermographer to be advanced within or immediately adjacent to the field of view of the imaging system, permitting simultaneous acquisition and real-time display of images and temperature data of the same or substantially the same axial or angular locations within the vessel. This eliminates the need to correlate and couple imaging and thermography data prior to display. Accordingly, a medical practitioner may immediately investigate potential areas within the vessel susceptible of harboring vulnerable plaque using the real-time images and temperature data. As an alternative to thermographers, higher resolution imaging probes or wires may be advanced through the side exit port to characterize vulnerable plaque. These include, for example, Optical Coherence Tomography probes or wires.

As yet another embodiment, rather than having a side exit port, the catheter may comprise a distal exit port disposed at the distal end of the catheter through which a thermographer of the present embodiment may be advanced. The thermographer may comprise a shape memory wire that may, upon advancement past the distal exit port, be everted to dispose the distal working end of the thermographer in sensory proximity with the vessel wall and in the field of view of the proximally disposed imaging system.

A still further embodiment comprises a catheter having a phased-array IVUS imaging system and a plurality of thermographers that are circumferentially disposed about the catheter and affixed thereto so that the distal portions of the thermographers radially self-expand away from the catheter when a delivery sheath is proximally retracted. Radial expansion of the plurality of thermographers permits each thermographer to contact the interior wall of a patient's body lumen.

Embodiments of the present invention may comprise one or more thermographers adapted to obtain the ambient temperature within the vessel. These thermographers may be disposed, for example, on the distal end of catheters made in accordance with the present invention. Additional locations will be apparent to those of skill in the art. Relative temperature increase or decrease at the vessel wall may then be determined by subtracting out the ambient temperature within the vessel.

These embodiments are provided only for the purpose of illustration. Additional embodiments will be apparent to those skilled in the art and are included in the scope of the present invention.

Imaging and thermographic data preferably are coupled in order to facilitate identification of vulnerable plaque. Coupling may be achieved using position indication techniques, for example, using an IVUS pullback system that is modified to simultaneously monitor the position of both the imaging element and the thermographer. IVUS pullback systems are described, for example, in U.S. Pat. No. 6,290,675 to Vujanic et al., U.S. Pat. No. 6,275,724 to Dickinson et al., U.S. Pat. No. 6,193,736 to Webler et al., and PCT Publication WO 99/12474, all of which are incorporated herein by reference. Additionally, relative distances between imaging elements and thermographers on catheters comprising both are preferably obtained prior to introduction of such catheters within a patient's vasculature. Measurement of such relative distances is expected to facilitate correlation of imaging and thermographic data.

Imaging data and thermographic data, coupled using position indication techniques and measured relative distances, preferably are simultaneously graphically displayed, for example, on a standard computer monitor. The coupled data preferably is displayed in a separate, yet overlaid fashion so that a medical practitioner may rapidly correlate temperature measurements obtained at a given position within the patient's body lumen to images obtained at that position. Rapid correlation is expected to simplify, expedite and increase the accuracy of vulnerable plaque identification, as well as facilitate plaque stabilization. The overlaid data may also be combined by, for example, color-coding the imaging data to represent temperature.

It is expected that additional data for additional vessel parameters also may be obtained, coupled and provided in the graphical display, for example, palpography, pressure, and pH data. Blood flow imaging, as described, for example, in U.S. Patent Nos. 5,453,575 and 5,921,931 to O'Donnell et al., both of which are incorporated herein by reference, also may be provided.

In accordance with another aspect of the present invention, data for a vessel parameter may be displayed on an interactive 3-dimensional graph in which the data may be provided as a function of axial and angular position within the vessel. Selection of a particular value of one of the variables (e.g., vessel parameter data, axial position or angular position) may prompt display of a 2-dimensional graph in which the coordinate axes comprise the remaining two variables, or display of an image of the associated cross-section or side-section having the vessel parameter data overlaid thereon.

Vessel parameter data also may be conditioned to facilitate rapid bulk testing to narrow the region(s) of the vessel that may require additional analysis. Such conditioning may include computation and display of average vessel parameter values for a particular cross-section or side-section of the vessel, gradients of the individual or average vessel parameter values, and/or accentuation of shifts in individual or average vessel parameter data.

Methods of using the apparatus of the present invention also are provided.

### BRIEF DESCRIPTION OF THE DRAWINGS

Further features of the invention, its nature and various advantages, will be more apparent from the following detailed description of the preferred embodiments, taken in conjunction with the accompanying drawings, in which like reference numerals apply to like parts throughout, and in which:

FIG. 1 is a schematic cut-away view of a prior art phased-array IVUS catheter;

FIG. 2 is a schematic cut-away view of a prior art rotational IVUS catheter;

FIGS. 3A and 3B are schematic side views of a prior art thermography catheter having a plurality of thermocouples, and shown in a collapsed delivery configuration and an expanded deployed configuration, respectively;

FIG. 4 is a schematic cut-away view of a prior art thermography catheter having a side-viewing infrared thermographer;

FIG. 5 is a schematic side view of a prior art thermography catheter having a steerable distal region with a thermocouple;

FIG. 6A is a schematic side view of a first embodiment of a catheter in accordance with the principles of the present invention having an imaging element and a thermographer;

FIG. 6B is a schematic side view of an alternative embodiment of the catheter of FIG. 6A in accordance with the principles of the present invention having an imaging element and a thermographer;

FIG. 7 is a schematic cut-away view of a second embodiment of apparatus of the present invention having an imaging element and a thermographer;

FIGS. 8A and 8B are schematic cut-away side views of an alternative embodiment of the apparatus of FIG. 7;

FIG. 9 is a schematic side view of a fourth embodiment of apparatus in accordance with the present invention having an optional stabilization element, as well as an optional embolic protection device;

FIG. 10 is a schematic side view of a fifth embodiment of the present invention having an alternative stabilization element;

FIGS. 11A-11C are schematic cut-away side views of a sixth embodiment of a catheter of the present invention having at least one side exit port for advancement of a thermographer;

FIGS. 12A-12D are schematic side views and cross-sectional views of alternative embodiments of the present invention having an evertable thermographer;

FIGS. 13A and 13B are schematic side views of a further alternative embodiment of the present invention having self-expanding thermographers;

FIGS. 14A and 14B are schematic side views, partially in section, of the apparatus of FIG. 7 disposed at a target site within a patient's vessel, illustrating a method of using the apparatus of the present invention;

FIGS. 15A and 15B are schematic views of graphical user interfaces that display imaging and thermographic data, respectively, obtained, for example, via the method of FIGS. 14, with the thermographic data of FIG. 15B obtained along side-sectional view line A-A of FIG. 15A;

FIG. 16 is a schematic view of a graphical user interface that couples and simultaneously displays imaging and thermographic data obtained along a cross-section of the patient's vessel;

FIG. 17 is a schematic view of an alternative graphical user interface that simultaneously displays coupled imaging and thermographic data along side-sectional view line B-B of FIG. 16;

FIG. 18 is a schematic perspective view of an illustrative vessel having a vulnerable plaque;

FIG. 19 is a schematic view of a graphical user interface that displays illustrative thermographic data corresponding to the vessel of FIG. 18 as a function of axial and angular position within a patient's vessel;

FIG. 20 is a schematic view of a graphical user interface that displays illustrative thermographic data corresponding to the vessel of FIG. 18 as a function of angular position;

FIG. 21 is a schematic view of a graphical user interface that displays gradients of average summation values of thermography data at multiple cross-sections of the vessel of FIG. 18;

FIG. 22 is a schematic cut-away view of a alternative embodiment of apparatus of the present invention comprising a forward-looking imaging element and a forward-looking infrared element;

FIG. 23 is a schematic perspective view illustrating construction of the phased-array IVUS imaging element of the apparatus of FIG. 22;

FIG. 24 is a schematic cross-sectional view of the infrared element of the apparatus of FIG. 22;

FIG. 25 is a side view, partially in section, illustrating a method of using the apparatus of FIG. 22 at a vascular occlusion within a patient;

FIG. 26 is a schematic side-sectional view of a further alternative embodiment of apparatus of the present invention comprising a radially-viewing imaging element and a radially-viewing infrared element;

FIG. 27 is a side-sectional view illustrating a method of using the apparatus of FIG. 26 at a stenosed region within a patient's vasculature;

FIG. 28 is a schematic side view of yet another alternative embodiment of the present invention comprising a radially-viewing imaging element and a single fiber side-looking infrared element;

FIGS. 29A and 29B are cross-sectional views illustrating a method of using and aligning the apparatus of FIG. 28 at a stenosed region within a patient's vasculature;

FIG. 30 is a side view of an alternative embodiment of the apparatus of claim 28; and

FIG. 31 is a schematic view of a graphical user interface that provides both cross-sectional and longitudinal side-sectional views of a vessel segment of interest, wherein thumbnail cross-sectional views are provided for reference at points along the longitudinal side-sectional view.

### DETAILED DESCRIPTION OF THE INVENTION

The present invention relates to methods and apparatus for identifying and stabilizing vulnerable plaque. More particularly, the present invention relates to specialized catheters having both an imaging element and a thermographer for improved identification of vulnerable plaque. Apparatus of the present invention may in addition include an optional stabilization element for stabilizing the plaque.

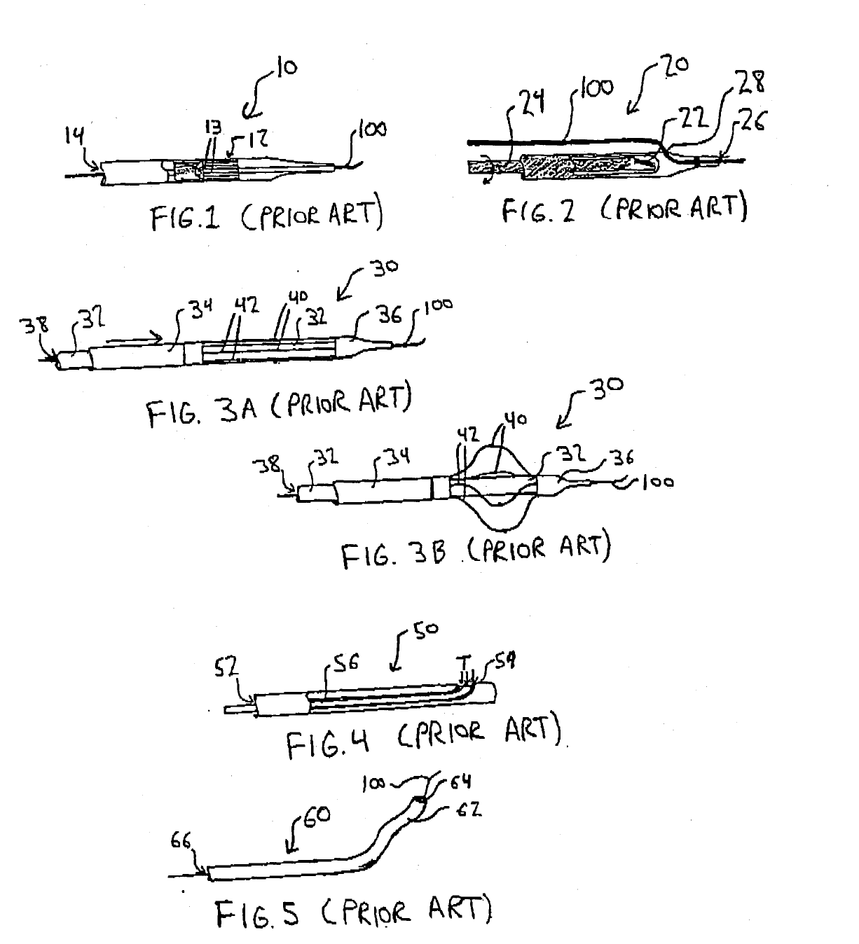

With reference to FIG. 1, a prior art phased-array Intravascular Ultrasound (“IVUS”) catheter is described. Catheter 10 comprises phased-array ultrasound transducer 12 having a plurality of discrete ultrasound elements 13. Catheter 10 further comprises guide wire lumen 14, illustratively shown with guide wire 100 disposed therein. Catheter 10 also may comprise multiplexing circuitry, amplifiers, etc., per se known, which may be disposed on and/or electrically coupled to catheter 10. Transducer array 12 of catheter 10 is electrically coupled to an imaging system (not shown), per se known, that provides excitation waveforms to the transducer array, and interprets and displays data received from the array.

FIG. 2 depicts a prior art rotational IVUS catheter. Catheter 20 comprises ultrasound transducer 22 disposed on a distal region of rotatable drive cable 24. Drive cable 24 is proximally coupled to a driver (not shown), e.g. an electric motor, for rotating the drive cable and ultrasound transducer 22, thereby providing transducer 22 with a 360° view. Catheter 20 further comprises guide wire lumen 26 that opens in side port 28 distally of transducer 22. Guide wire 100 is illustratively disposed within lumen 26. As with transducer array 12 of catheter 10, transducer 22 of catheter 20 is electrically coupled to an imaging system (not shown), per se known, that provides excitation waveforms to the transducer, and interprets and displays data received from the transducer.

As discussed hereinabove, it has been shown that sub-intimal lipid pools at the site of plaque, as well as the eccentricity of the plaque, are key indicators of vulnerable plaque susceptible to rupture. It has also been shown that IVUS may be used to determine the eccentricity of plaque, as well as to identify echolucent zones, which are indicative of lipid-rich cores. However, achieving proper identification of vulnerable plaque via IVUS or any of a host of other advanced imaging modalities (e.g. Magnetic Resonance Imaging or Optical Coherence Tomography) may require a significant degree of skill, training and intuition on the part of a medical practitioner.

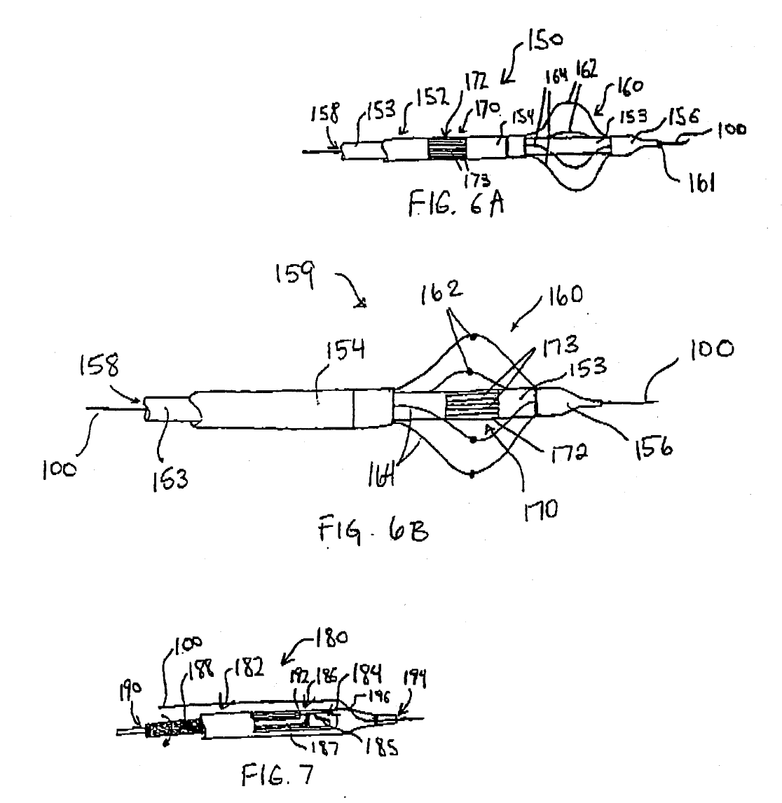

With reference now to FIG. 3, a prior art thermography catheter is described. Catheter 30 comprises outer tube 34 coaxially disposed about inner tube 32. Inner tube 32 comprises distal tip 36 and guide wire lumen 38, in which guide wire 100 is illustratively disposed. Catheter 30 further comprises a plurality of thermocouples 40 disposed near its distal end. Each thermocouple comprises a wire 42 coupled proximally to the distal end of outer tube 34 and distally to distal tip 36 of inner tube 32. The proximal and distal ends of each wire 42 are further electrically coupled to a processor (not shown) that captures and translates voltages generated by thermocouples 40 into temperature values, for example, via known calibration values for each thermocouple.

As seen in FIG. 3, catheter 30 is expandable from the collapsed delivery configuration of FIG. 3A to the expanded deployed configuration of FIG. 3B, by advancing outer tube 34 with respect to inner tube 32. Such advancement causes thermocouples 40 to protrude from catheter 30 so that the thermocouples may contact the interior wall of a patient's body lumen. Catheter 30 is adapted for intravascular delivery in the collapsed configuration of FIG. 3A, and is adapted for taking temperature measurements at a vessel wall in the expanded configuration of FIG. 3B.

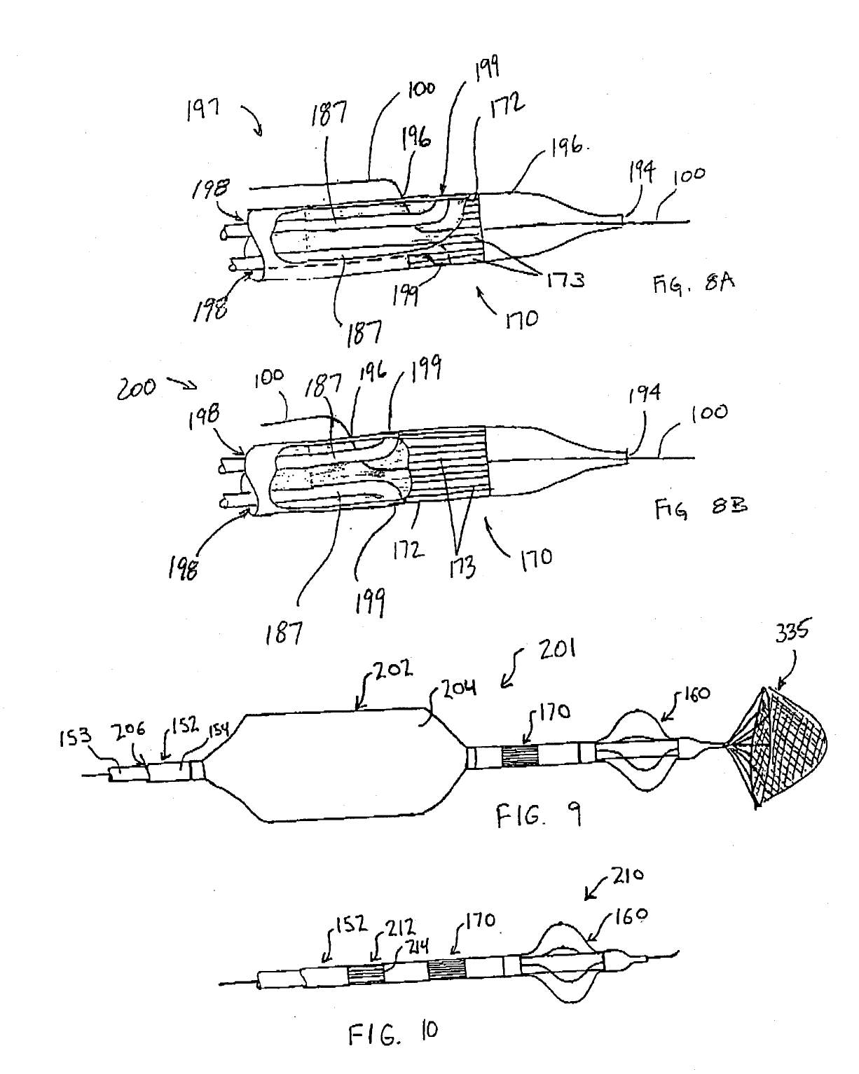

Referring to FIG. 4, another prior art thermography catheter is described. Catheter 50 comprises lumen 52, which extends from a proximal end of catheter 50 to distal side port 54. Fiber optic 56 is disposed within lumen 52 and is proximally coupled to an infrared thermography system (not shown). Catheter 50 thereby comprises a side-viewing fiber optic thermography catheter capable of measuring ambient temperature T near distal side port 54.

By disposing side port 54 of catheter 50 within a patient's body lumen, the temperature of the patient's body lumen may be measured to facilitate identification of vulnerable plaque. However, a significant drawback of catheter 50 for identification of vulnerable plaque is that fiber optic 56 has only a limited field of view, and vulnerable plaque is typically eccentric, i.e. occurs predominantly on one side of a vessel. Thus, if side port 54 of catheter 50 were not rotated to the side of the vessel afflicted with vulnerable plaque build-up, it is expected that the ambient temperature T measured with catheter 50 would not reflect the presence of vulnerable plaque.

With reference to FIG. 5, yet another prior art thermography catheter is described. Catheter 60 comprises steerable distal end 62 having thermistor 64 coupled thereto. Thermistor 64 is proximally attached to a processor (not shown) that converts measurements taken with thermistor 64 into temperature measurements. Catheter 60 further comprises guide wire lumen 66 having guide wire 100 illustratively disposed therein.

Distal end 62 of catheter 60 may be positioned against a patient's body lumen to provide temperature measurements where thermistor 64 contacts the body lumen. However, a significant drawback of catheter 60 is that thermistor 64 only provides temperature measurements at a single point at any given time. It is therefore expected that eccentric vulnerable plaque will be difficult to identify with catheter 60, especially if distal end 62 of catheter 60 is disposed against the unaffected, or mildly affected, side of a patient's vessel suffering from eccentric vulnerable plaque.

Although thermography is a promising new technique for identifying vulnerable plaque, the thermography devices described hereinabove have several drawbacks. Since thermography doesn't provide image data, it is expected that medical practitioners will have difficulty determining proper locations at which to use a thermographer in order to characterize plaque type. Thus, secondary, stand-alone imaging apparatus may be required in order to adequately identify and characterize plaque. Requiring separate imaging and thermography apparatus is expected to increase complexity, time and cost associated with identifying vulnerable plaque. Additionally, thermography provides no indication of the eccentricity of a plaque or of the presence or magnitude of lipid pools disposed in the plaque, both of which have been shown to indicate the presence of vulnerable plaque.

With reference now to FIG. 6A, a first embodiment of apparatus in accordance with the present invention is described that provides both an imaging element and a thermographer in a single device. By providing both imaging and thermography in a single device, the present invention combines positive attributes of stand-alone imaging systems and stand-alone thermographers described hereinabove, while reducing previously-described drawbacks associated with such stand-alone systems. Apparatus 150 of FIG. 6A comprises catheter body 152, thermographer 160 and imaging element 170.

Catheter body 152 comprises outer tube 154 coaxially disposed about inner tube 153. Inner tube 153 comprises distal tip 156 and guide wire lumen 158, in which guide wire 100 is illustratively disposed. Thermographer 160 comprises a plurality of thermocouples 162. Any number of thermocouples 162 may be provided. Each thermocouple comprises a wire 164 coupled proximally to the distal end of outer tube 154 and distally to distal tip 156 of inner tube 153. The proximal and distal ends of each wire 164 are further electrically coupled to a processor (not shown) that captures and translates voltages generated by thermocouples 162 into temperature values, for example, via known calibration values for each thermocouple.

Thermographer 160 optionally may also comprise thermosensor 161 disposed, for example, on distal tip 156. Thermosensor 161 may be used to determine ambient temperature within a body lumen such as a blood vessel. This ambient temperature may be subtracted from temperature measurements obtained with thermocouples 162 so that changes in temperature, as opposed to absolute temperature, at a vessel wall may be examined.

Imaging element 170 comprises phased-array ultrasound transducer 172 having a plurality of discrete ultrasound elements 173. Imaging element 170 optionally may comprise multiplexing circuitry, flexible circuitry or substrates, amplifiers, etc., per se known, which may be disposed on and/or electrically coupled to apparatus 150. Transducer array 172 of imaging element 170 is electrically coupled to an imaging system (not shown), per se known, that provides excitation waveforms to the transducer array, and interprets and displays data received from the array. The imaging system coupled to imaging element 170 and the processor coupled to thermographer 160 are preferably combined into a single data acquisition and analysis system (not shown) for capturing and interpreting data received from apparatus 150.

As with catheter 30 of FIG. 3, apparatus 150 is expandable from a collapsed delivery configuration to the expanded deployed configuration of FIG. 6A, by advancing outer tube 154 of catheter body 152 with respect to inner tube 153. Such advancement causes thermocouples 162 of thermographer 160 to protrude from catheter body 152 so that the thermocouples may contact the interior wall of a patient's body lumen. Apparatus 150 is adapted for intravascular delivery in the collapsed configuration, and is adapted for taking temperature measurements at a vessel wall in the expanded configuration. Imaging via imaging element 170 may be achieved in either the collapsed delivery configuration or the expanded deployed configuration, thereby facilitating positioning of apparatus 150 at a stenosed region within a patient's vessel.

Thermographer 160 comprises multiple thermography sensors, illustratively in the form of thermocouples 162, disposed radially about catheter body 152. Temperature measurements obtained from these sensors may be displayed graphically as a 2-dimensional map or image, for example, as a cross-sectional temperature profile within a patient's vessel. Such a cross-sectional temperature profile may be compared with a cross-sectional image of the vessel obtained at the same location, for example, via imaging element 170. Correlation of imaging and thermography data may be facilitated by determining the distance between imaging element 170 and thermographer 160 prior to use. By advancing or retracting catheter body 152, correlated, 2-dimensional temperature and imaging data may be extended to 3-dimensions. Translation of catheter body 152 may be achieved, for example, using position indication techniques and/or a pullback system, per se known. Illustrative methods and apparatus for displaying thermographic and imaging data are provided hereinbelow with respect to FIGS. 14-21.

Apparatus 150 is expected to provide significant advantages over prior art, stand-alone imaging and thermography catheters, such as catheters 10 and 30, used either alone or in combination. Specifically, apparatus 150 is expected to decrease the complexity of obtaining both temperature and imaging data at a target site, as well as to facilitate correlation of such data. Additionally, apparatus 150 is expected to reduce the cost of obtaining both temperature and imaging data, as compared to providing both a stand-alone imaging system and a stand-alone thermography system.

Since vascular lumens commonly afflicted with vulnerable plaque, such as the coronary arteries, are often very small, it is expected that difficulty may be encountered while trying to simultaneously position separate imaging and thermography catheters at the site of vulnerable plaque; furthermore, a stand-alone thermography catheter may block imaging of portions of the vessel wall. Apparatus 150 overcomes these drawbacks. Additionally, apparatus 150 is expected to reduce the skill required on the part of a medical practitioner to identify vulnerable plaque via IVUS, by providing a secondary indication of vulnerable plaque in the form of temperature measurements. Likewise, apparatus 150 is expected to increase the likelihood of proper vulnerable plaque identification via thermography, by providing a secondary indication of vulnerable plaque in the form of IVUS imaging that allows examination of plaque eccentricity and echogenicity. Additional advantages of the present invention will be apparent to those of skill in the art.

An alternative embodiment of catheter 150 of FIG. 6A is illustrated in FIG. 6B. As with catheter 150, catheter 159 also comprises catheter body 152, thermographer 160 comprising a plurality of thermocouples 162, and imaging element 170 comprising phased-array ultrasound transducer 172. The difference between catheter 159 and catheter 150 resides in the configuration of thermographer 160 with respect to imaging element 170. Specifically, while thermographer 160 of catheter 150 is disposed longitudinally distant from imaging element 170, thermocouples 162 may be disposed at the same axial location as imaging element 170.

In addition to the advantages discussed above with reference to catheter 150, catheter 159 provides the further advantage of disposing thermocouples 162 within the field of view of phased-array ultrasound transducer 172. This facilitates simultaneous acquisition, real-time viewing and correlation of both temperature and imaging data at the same axial and/or angular positions within vessel V, thereby eliminating the need to correlate and couple the temperature and imaging data prior to display. In particular, a medical practitioner may be able to view a real-time, cross-sectional image of the vessel with the temperature data instantly overlaid thereon. This permits the medical practitioner to immediately acquire knowledge of, and investigate potential areas within, the vessel suspected of harboring vulnerable plaque.

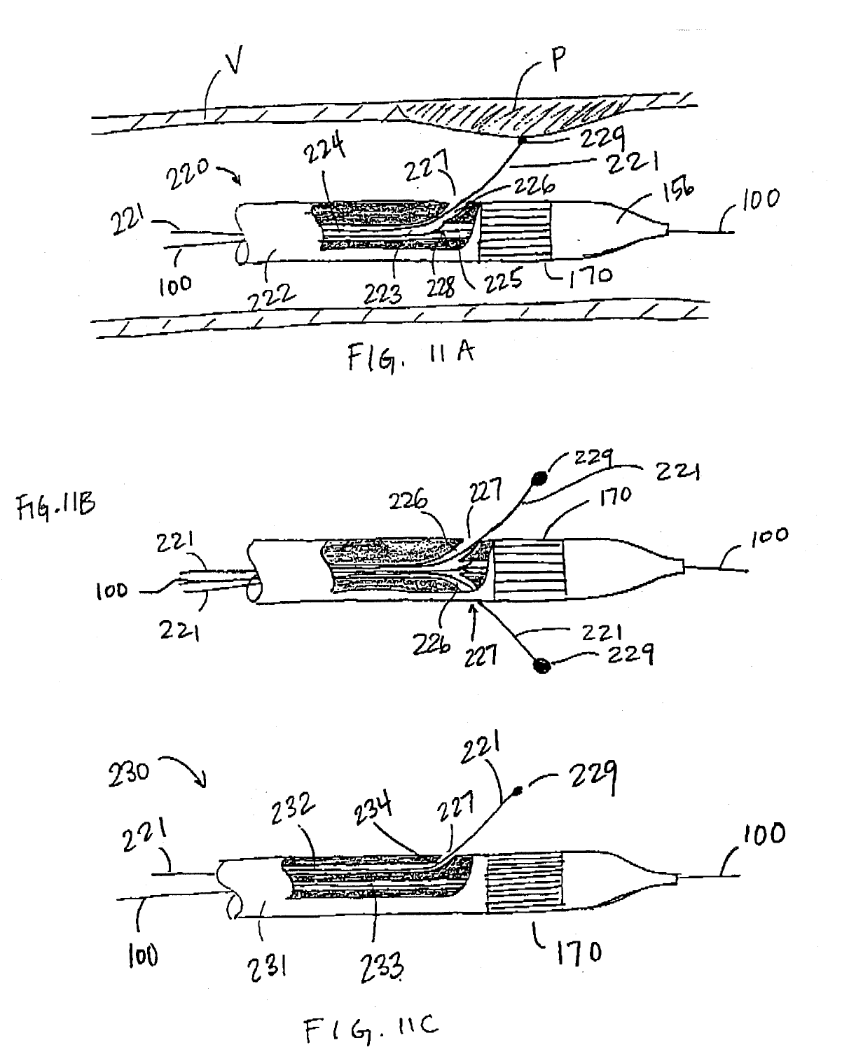

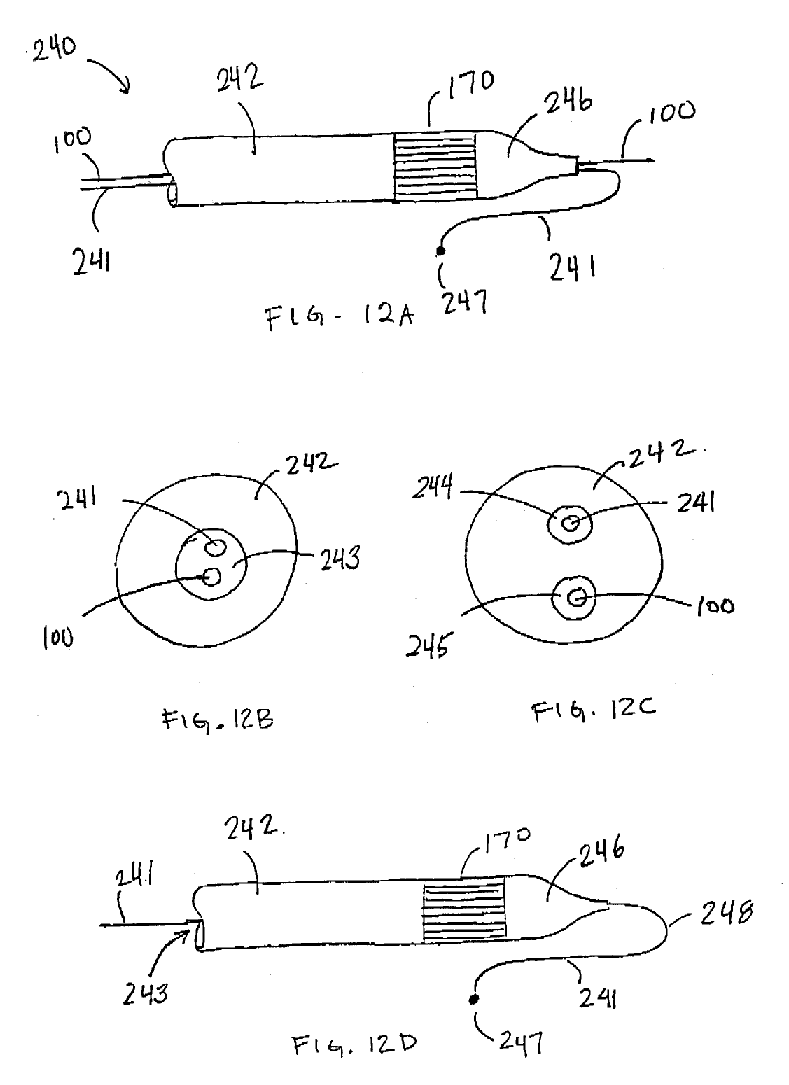

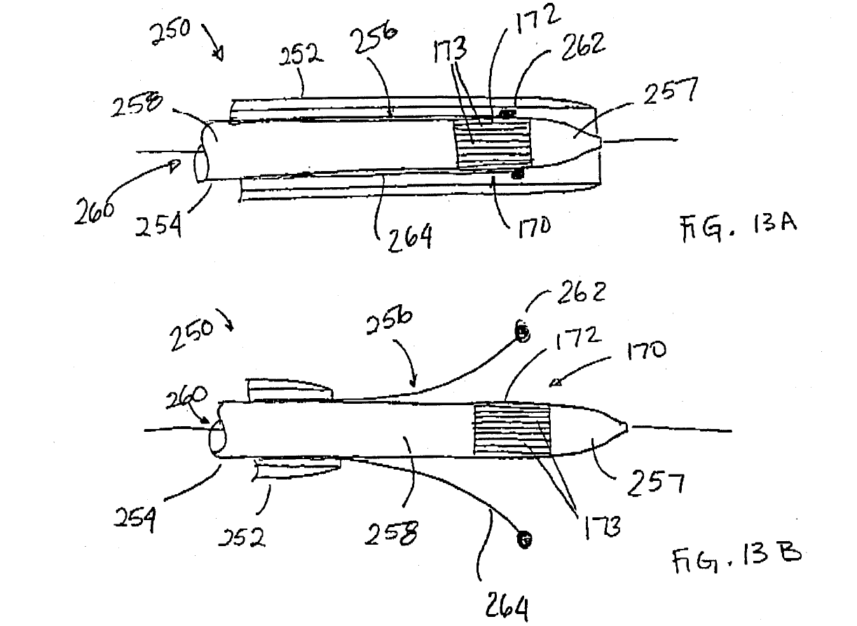

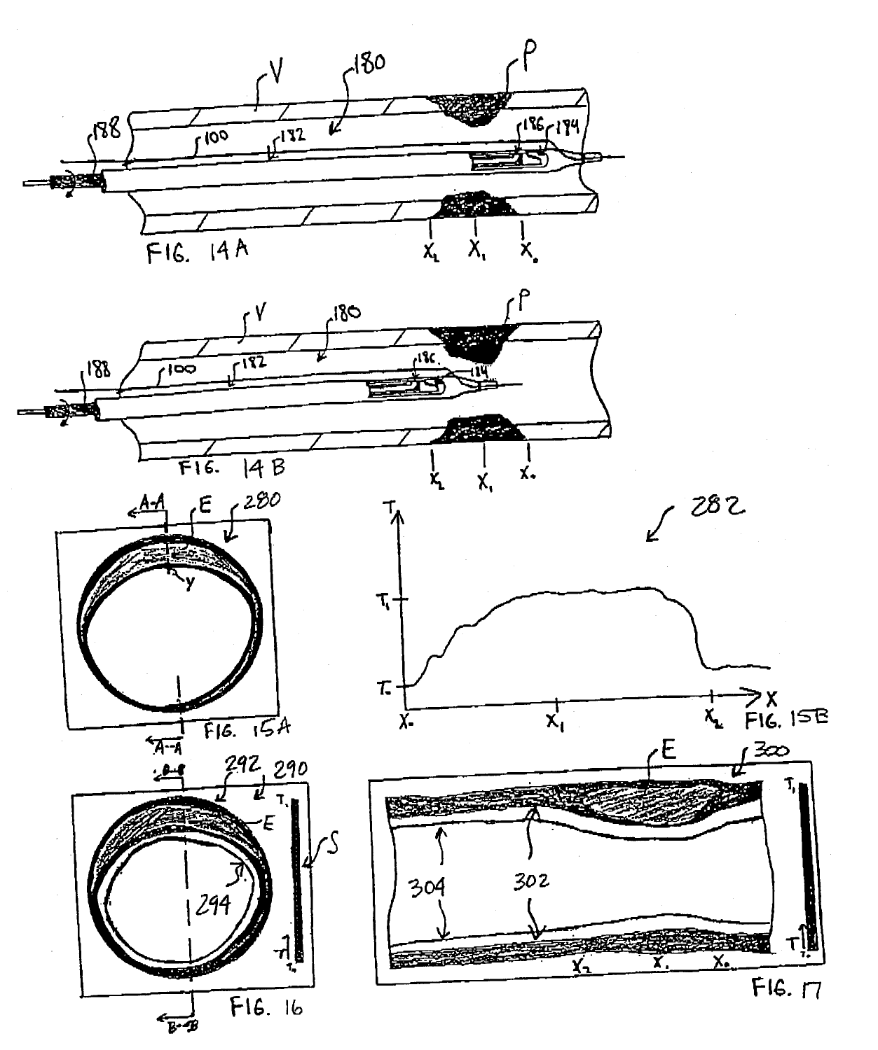

Referring now to FIG. 7, a second embodiment of apparatus in accordance with the present invention in described. Apparatus 180 comprises catheter 182 having imaging element 184 and thermographer 186. Imaging element 184 comprises a rotational IVUS imaging element, and thermographer 186 comprises a rotational infrared thermographer.

Catheter 182 further comprises rotatable drive cable 188 having lumen 190 that distally terminates at side port 192. Catheter 182 still further comprises guide wire lumen 194 that opens in side port 196 distally of drive cable 188. Guide wire 100 is illustratively shown disposed in lumen 194.

Thermographer 186 of catheter 182 comprises fiber optic 187 disposed within lumen 190 of drive cable 188. Imaging element 184 of catheter 182 comprises ultrasound transducer 185 disposed on rotatable drive cable 188. Drive cable 188 is proximally coupled to a driver (not shown), e.g. an electric motor, for rotating the drive cable, as well as ultrasound transducer 185 of imaging element 184 and fiber optic 187 of thermographer 186, thereby providing imaging element 184 and thermographer 186 with a 360° view. It will be evident to one of ordinary skill in the art that fiber optic 187 may comprise two or more fibers adjacently disposed, at least one fiber for transmitting a signal and at least one fiber for receiving the transmitted signal.

As with transducer 22 of catheter 20, transducer 185 is electrically coupled to an imaging system (not shown), per se known, that provides excitation waveforms to the transducer, and interprets and displays data received from the transducer. Likewise, as with fiber optic 56 of catheter 50, fiber optic 187 is proximally coupled to an infrared thermography system (not shown). Preferably, the imaging system of imaging element 184, the infrared thermography system of thermographer 186, and the driver coupled to drive cable 188, are combined into a single data acquisition and analysis system (not shown) for capturing and interpreting data received from apparatus 180. Alternatively, a subset of these elements may be combined. Determination of the distance between imaging element 184 and thermographer 186 prior to use is expected to facilitate correlation of imaging and thermography data.

Apparatus 180 provides many of the advantages described hereinabove with respect to apparatus 150. Additionally, as compared to infrared thermography catheter 50, described hereinabove with respect to FIG. 4, thermographer 186 of apparatus 180 provides significantly enhanced thermographic capabilities. Specifically, by coupling thermographer 186 to rotatable drive cable 188, thermographer 186 is capable of providing a full circumferential temperature profile along the interior wall of a patient's body lumen, without necessitating potentially inaccurate manual rotation of the infrared thermographer by a medical practitioner. A stand-alone, rotatable infrared thermography catheter (not shown), similar to apparatus 180 but without imaging capabilities, is contemplated and is included in the scope of the present invention.

In an alternative embodiment of apparatus 180 of FIG. 7, imaging element 184, comprising a rotational IVUS imaging element, is replaced with imaging element 170 of FIG. 6. Imaging element 170 comprises phased-array ultrasound transducer 172 having plurality of discrete ultrasound elements 173. Apparatus 197 further comprises plurality of lumens 198 that distally terminate at plurality of side ports 199.

Plurality of side ports 199 are disposed on a lateral surface of apparatus 197 at a longitudinal position that is coincident with that of ultrasound transducer 172 so that the circumferential orientation of discrete ultrasound elements 173 is interrupted at regular angular intervals to expose fiber optics 187 disposed within lumens 198. This permits apparatus 197 to simultaneously acquire both circumferential temperature and imaging profiles at the same axial position within a patient's body lumen. As will be apparent to those of skill in the art, the plurality of lumens and side ports may comprise any number of lumens and side ports, including a single lumen and side port.

To provide a full circumferential image profile without the attendant interruptions of ultrasound elements 173, side ports 199 may be shifted to a longitudinal position immediately adjacent to imaging element 170, as illustrated in FIG. 8B. While this configuration does not permit simultaneous acquisition of temperature and imaging data at exactly the same axial position within a patient's body lumen, apparatus 200 allows simultaneous acquisition at substantially the same axial position. Specifically, the temperature data acquired by apparatus 200 corresponds to image data of the body lumen just proximal to the field of view of the imaging element. Accordingly, a medical practitioner may still obtain real-time viewing and correlation of both temperature and imaging data at approximately the same axial body lumen position for investigation of areas within the body lumen suspected of harboring vulnerable plaque.

In FIG. 8B, to facilitate correlation of temperature and imaging data at exactly the same axial position post-acquisition, the distance between side exit ports 199 and imaging element 170 preferably are provided or measured. The offset between the side ports and the imaging element may be subtracted out, for example, during data processing. Placing side exit ports 199 immediately adjacent imaging element 170 is expected to reduce artifacts within images obtained with the imaging element caused by placement of thermographers directly within the plane of view of the imaging element.

With reference to FIG. 9, a fourth embodiment of apparatus in accordance with the present invention is described that includes an optional stabilization element, in addition to an imaging element and a thermographer. The stabilization element is adapted to stabilize vulnerable plaque, thereby providing vulnerable plaque identification and stablization in a single device. Apparatus 201 comprises all of the elements of apparatus 150, including catheter body 152, thermographer 160 and imaging element 170, and further comprises stabilization element 202.

Stabilization element 202 comprises inflatable balloon 204. Balloon 204 is inflatable from a collapsed delivery configuration to the deployed configuration of FIG. 9 by suitable means, for example, via an inflation medium injected into the balloon through annulus 206 formed between the inner wall of outer tube 154 and the outer wall of inner tube 153 of catheter body 152. Additional inflation techniques will be apparent to those skilled in the art.

It is expected that, once vulnerable plaque has been identified in a patient's vessel via thermographer 160 and/or imaging element 170, stabilization element 202 may be positioned at the location of the identified vulnerable plaque. Stabilization element 202 may then be deployed, i.e. balloon 204 may be inflated, at the site of vulnerable plaque to stabilize the plaque, for example, by compressing, rupturing, scaffolding and/or sealing the plaque in the controlled environment of a catheterization laboratory. In addition to balloon 204, stabilization element 202 may be provided with additional stabilization elements (not shown), for example, a stent, a covered stent, a stent graft, a coated stent or a drug-eluting stent, to further enhance stabilization of vulnerable plaque. Additional stabilization elements will be apparent to those of skill in the art.

In order to facilitate identification and stabilization of vulnerable plaque, the distances between stabilization element 202, thermographer 160 and imaging element 170 are preferably provided or measured. Furthermore, the distances between the imaging, thermography and optional stabilization elements of all embodiments of the present invention are preferably provided or measured. This facilitates coupling of thermographic and imaging data, as well as proper positioning of optional stabilization elements.

Providing vulnerable plaque identification and stabilization elements in a single device, in accordance with the principles of the present invention, provides all of the benefits of apparatus 150 described hereinabove, as well as the additional advantage of not having to provide stand-alone apparatus for plaque stabilization. This, in turn, is expected to decrease the cost, time and complexity associated with identifying and stabilizing vulnerable plaque, as well as to decrease the crossing profile of such apparatus, as compared to stand-alone apparatus used concurrently. Further still, providing identification and stabilization in a single device is expected to simplify accurate placement of stabilization elements at the site of identified vulnerable plaque.

Referring now to FIG. 10, a fifth embodiment of the present invention having an alternative vulnerable plaque stabilization element, is described. Apparatus 210 comprises all of the elements of apparatus 150, including catheter body 152, thermographer 160 and imaging element 170, and further comprises stabilization element 212. Stabilization element 212 comprises therapeutic ultrasound transducer 214, which is capable of resonating at, and transmitting, therapeutic ultrasound frequencies. Transducer 214 may comprise a single element or an array of elements. Transducer 214 is attached to an excitation unit (not shown) capable of causing resonance within the transducer. The excitation unit is preferably combined with the imaging system (not shown) of imaging element 170.

Therapeutic ultrasound frequencies, at which therapeutic transducer 214 preferably is capable of resonating and transmitting, are typically described as low frequencies, for example, frequencies below 10,000,000 Hertz, or 10 Megahertz (“MHz”), and even more preferably frequencies below about 500,000 Hertz, or 500 Kilohertz (“kHz”). Conversely, transducer array 172 of imaging element 170 preferably is capable of resonating at, and transmitting, imaging ultrasound frequencies. Imaging ultrasound frequencies are typically described as high frequencies, for example, frequencies above about 10 Megahertz (“MHz”). These frequencies are provided only for the sake of illustration and should in no way be construed as limiting.

It is expected that, once vulnerable plaque has been identified in a patient's vessel via thermographer 160 and/or imaging element 170, stabilization element 212 may be positioned at the location of the identified plaque and activated, i.e. ultrasound transducer 214 may provide therapeutic ultrasound waves, to stabilize the plaque, for example, by compressing, rupturing, and/or sealing the plaque in the controlled environment of a catheterization laboratory. As with apparatus 201, the distances between stabilization element 212, thermographer 160 and imaging element 170 are preferably provided or measured in order to facilitate vulnerable plaque identification, as well as positioning of stabilization element 212 prior to. activation.

In addition to therapeutic ultrasound transducer 214, stabilization element 212 may be provided with additional stabilization elements (not shown), for example, contrast, tissue-tag or therapeutic agents, such as drug capsules, that rupture and are released upon exposure to ultrasound waves generated by therapeutic ultrasound transducer 214. Additional stabilization elements will be apparent to those of skill in the art. Apparatus 210 is expected to provide many of the benefits described hereinabove with respect to apparatus 150 and apparatus 201.

As yet another embodiment of the present invention, apparatus may be provided in which imaging element 170 and stabilization element 212 of apparatus 210 are replaced with a single ultrasonic transducer array that is capable of transmitting multiple frequencies suited to both ultrasonic imaging and ultrasonic therapy, thereby providing both vulnerable plaque imaging and stabilization in a single element. Techniques for providing an ultrasound transducer capable of resonating at multiple frequencies are provided, for example, in U.S. Pat. No. 5,906,580 to Kline-Schoder et al., as well as U.S. Pat. No. 5,581,144 to Corl et al., both of which are incorporated herein by reference.

Referring to FIG. 11A, a sixth embodiment of the present invention is described. Apparatus 220 comprises functional measurement wire 221 and catheter 222 having imaging element 170. Wire 221 preferably comprises a thermographer such as a thermocouple, thermistor, or fiber optic infrared thermographer, but may comprise other diagnostic devices to measure, for example, pressure, flow velocity, pH or tissue composition. Further alternatives may include a secondary imaging device that provides a more detailed view than IVUS imaging element 170, such as an Optical Coherence Tomography apparatus, high frequency ultrasound transducer, Near Infrared Spectroscopy fiber optic, or Magnetic Resonance Imaging apparatus, or may comprise a stabilization device such as an ablation device, therapeutic ultrasound transducer, drug delivery device, therapeutic agent and the like for local delivery to vulnerable plaque P.

Catheter 222 further comprises bifurcated lumen 223 having proximal portion 224 that branches into distal portion 225 and bifurcated portion 226. Proximal portion 224 extends to the proximal end of catheter 222, while distal portion 225 extends through distal end 156. Bifurcated portion 226 terminates at side port 227 disposed on a lateral face of catheter 222. Adjacent the junction of proximal portion 224, distal portion 225 and bifurcated portion 226, uni-directional valve 228 is disposed within distal portion 225 to prevent advancement of thermographer wire 221 into distal portion 225 while permitting advancement of catheter 222 over guide wire 100. Guide wire 100 is illustratively shown disposed within proximal portion 224 and distal portion 225, whereas wire 221 traverses proximal portion 224 and bifurcated portion 226.

Advantageously, bifurcated portion 226 may be curved to direct advancement of wire 221 so that distal working tip 229 of wire 221 may be advanced into the field of view of imaging element 170, which is disposed distal to side exit port 227. Similar to catheter 159 of FIG. 6B, this facilitates simultaneous acquisition, real-time viewing and association of both temperature and imaging data respectively obtained by functional measurement wire 221 and imaging element 170 at the same axial and/or angular positions within vessel V, thereby eliminating the need to correlate and couple the temperature and imaging data prior to display. This permits a medical practitioner to view a real-time, cross-sectional image of the vessel with the associated temperature data overlaid thereon in real time. Furthermore, using the real-time images provided by imaging element 170 as a visual guide, wire 221 may be advanced into the field of view of imaging element 170, and a medical practitioner may steer working tip 229 to a particular location of interest within vessel V for data acquisition, for example by rotating catheter 222 and/or wire 221.

In accordance with another aspect of the present invention, bifurcated portion 226 may be curved to direct disposition of working tip 229 of wire 221 in sensory proximity with (i.e., contacting or adjacent to) target vascular tissue that is suspected of harboring vulnerable plaque P. This is especially significant since a variety of working tips 229 may require contact or close proximity with the vessel wall to obtain accurate or useful measurements. Such working tips include, for example, thermocouples and Optical Coherence Tomography probes (which may be unable to visualize through blood). Furthermore, pursuant to fluid dynamics theory, blood flow velocity is slowest near the wall of vessel V. Thus, positioning working tip 229 at or near the wall is expected to reduce unwanted migration of the tip due to pressure applied to the tip by blood flowing through the vessel.

Alternatively, bifurcated portion 226 may be curved to direct advancement of wire 221 so that distal working tip 229 is disposed in an axial position immediately adjacent to the field of view of imaging element 170, and a radial position in sensory proximity with target vascular tissue. This reduces potentially undesirable imaging artifacts, such as incorporation of wire 221 and distal working tip 229 within the acquired images, that may result from advancement of distal working tip 229 within the field of view of imaging element 170. Advantageously, a medical practitioner may still simultaneously obtain both temperature and imaging data at substantially the same axial position within a patient's body lumen, thereby permitting real-time viewing, analysis and/or diagnosis.

It will be evident to one of ordinary skill in the art that apparatus 220 may comprise more than one curved, bifurcated portion 226. Additional bifurcated portions may be provided and disposed to radiate from proximal portion 224, distally terminating at side exit ports 227 circumferentially disposed on a lateral face of catheter 222 (see FIG. 11B). The additional bifurcated portions may direct advancement of distal working tips 229 of additional wires 221 into or immediately adjacent to the field of view of imaging element 170. This permits a medical practitioner to simultaneously obtain full circumferential temperature and imaging profiles along the interior wall of a patient's body lumen.

Advantageously, apparatus 220 provides for optional advancement of functional measurement wire 221, without requiring such advancement. Many patients may not have regions within their vasculature that are suspected of harboring vulnerable plaque. For these patients, the added time, expense, etc., of thermography or other data collection in conjunction with IVUS or other imaging modalities may not be justified. Apparatus 220 allows for optional use of functional measurement wire 221, for example, only in patients suspected of harboring vulnerable plaque.

In accordance with yet another aspect of the present invention, functional measurement wire 221 may be proximally removed from apparatus 220 once temperature or other data has been obtained, and successively replaced with other diagnostic, secondary imaging, and/or stabilization devices, examples of which are provided above. This permits a medical practitioner to initially locate vulnerable plaque P by simultaneous temperature and visual confirmation, and then obtain additional data about and/or a more detailed image of the plaque, or provide localized delivery of stabilization devices, while simultaneously viewing the interior of the vasculature to direct advancement of wire 221 or the replacement device. In this manner, apparatus 220 may be used to perform successive, multi-functional applications without removal of catheter 222 from the vessel site of interest.

Alternatively, rather than having bifurcated lumen 223, apparatus 230, illustrated in FIG. 11C, may instead comprise catheter 231 having separate wire lumen 232 and guide wire lumen 233. As with apparatus 220 of FIG. 10A, wire lumen 232 permits thermographer wire 221 to exit catheter 231 via side port 227 disposed on a lateral face of catheter 231. Distal portion 234 of wire lumen 232 is curved to permit working tip 229 of steerable wire 221 to be advanced within or immediately adjacent to the field of view of imaging element 170 and disposed in sensory proximity with (i.e., contacting or adjacent to) target vascular tissue that is suspected of harboring vulnerable plaque P. Furthermore, as with apparatus 220 in FIG. 11B, apparatus 230 may comprise additional wire lumens 232 disposed within catheter 231 that terminate at side exit ports circumferentially disposed on the lateral face thereof. Again, this allows additional functional measurement wires to be used in simultaneous acquisition of full circumferential temperature and imaging profiles.

Referring to FIGS. 12A-12C, an alternative embodiment of apparatus 220 and apparatus 230 of FIGS. 11 is described. Apparatus 240 comprises functional measurement wire 241 and catheter 242 having IVUS imaging element 170. Alternative imaging elements will be apparent. Wire 241 preferably comprises a thermographer, but also may comprise or be exchanged for other diagnostic, secondary imaging and/or stabilization devices.

Unlike apparatus 220 and apparatus 230 of FIG. 11, catheter 242 comprises either single lumen 243, as seen in FIG. 12B, or separate lumens 244 and 245, as seen in FIG. 12C, through which wire 241 may exit catheter 242 through distal end 246, instead of through side port 227 of FIG. 11. If catheter 242 comprises lumen 243, both functional measurement wire 241 and guide wire 100 may be advanced therethrough. If catheter 242 comprises separate lumens 244 and 245, wire 241 and guide wire 100 may be advanced through their respective lumens.

Functional measurement wire 241 of FIGS. 12A-C preferably comprises a shape memory alloy wire, e.g., a nickel titanium alloy. When wire 241 is extended from catheter 242, it adopts an everted curved shape that disposes distal working tip 247 of wire 241 within the field of view of imaging element 170, which is disposed proximally of distal end 246. In this everted configuration, a medical practitioner may rotate thermographer wire 241 and/or catheter 242 so that distal working tip 247 is in sensory proximity with target tissue P to obtain temperature (or other) data, using real-time images provided by imaging element 170 for visual guidance.

Once temperature data has been collected, wire 241 is retracted back into the lumen of catheter 242, thereby returning wire 241 to its non-everted shape. In the non-everted state, wire 241 may be removed from catheter 242 and optionally replaced with another diagnostic, secondary imaging, or stabilization device that also may be everted upon exiting distal end 246 to permit disposition of the distal working tip of the replacement device within the field of view of imaging element 170.

With reference to FIG. 12D, in an alternative embodiment of apparatus 240 of FIG. 12A, guide wire 100 may be eliminated. In this case, wire 241 initially may be inserted into vessel V as a straight wire. After catheter 242 is advanced along wire 241 to a general vessel location of interest, wire 241 may be extended to adopt an everted shape that disposes distal working tip 247 of guide wire 241 within the field of view of imaging element 107. Wire 241 optionally may be provided with a removable sheath (not shown) to maintain the wire in a straight configuration for use as a guide wire while catheter 242 is advanced thereover, at which time the sheath may be removed and wire 242 may resume its everted shape.

Catheter 242 then may be concurrently advanced with wire 241 in its everted shape along vessel V, using curve 248 of everted guide wire 241 as an atraumatic bumper. In this manner, a medical practitioner may be able to identify potential sites of vulnerable plaque P by simultaneously viewing both real-time imaging and temperature data respectively provided by imaging element 170 and wire 241 for the same axial and/or angular locations within vessel V.

As in preceding embodiments, wire 241 may adopt an everted curved shape that disposes distal working tip 247 of wire 241 immediately adjacent to the field of view of imaging element 170. This eliminates potentially undesirable imaging artifacts within the acquired images, such as the incorporation of wire 241 and working tip 247, and yet still permits a medical practitioner to simultaneously obtain both temperature and imaging data at substantially the same axial position along a patient's body lumen for real-time viewing, analysis, and/or diagnosis.

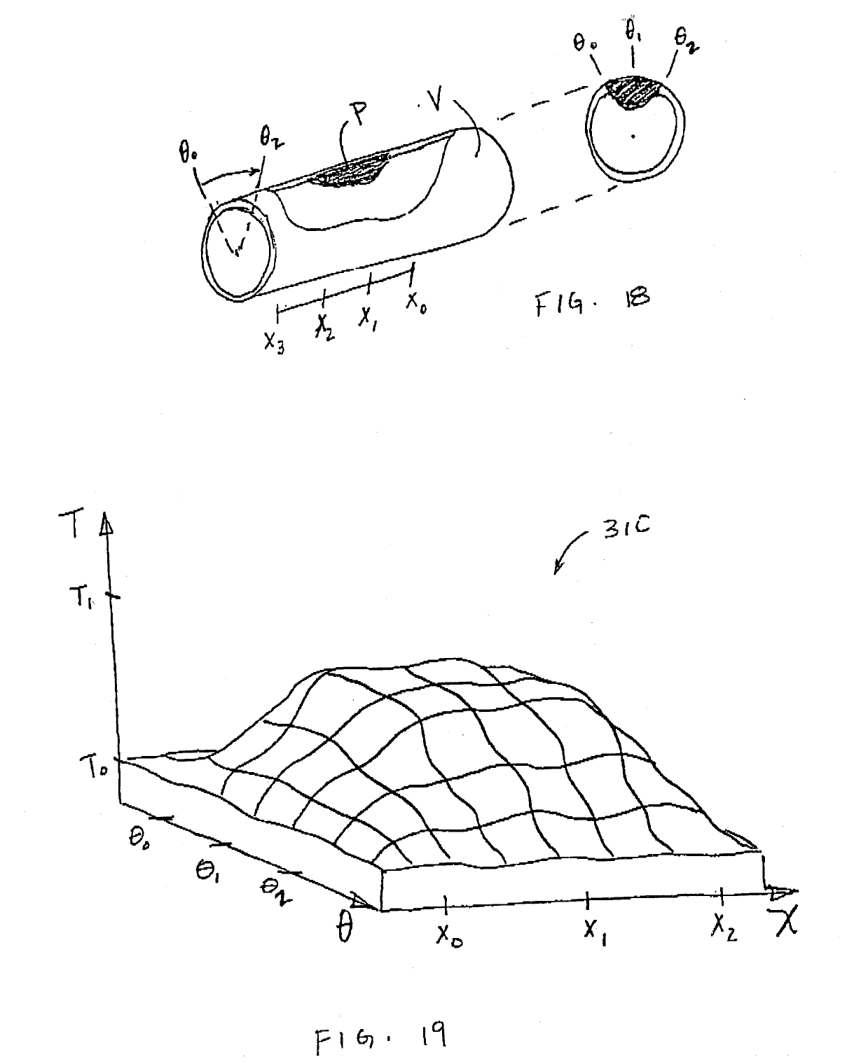

Referring now to FIG. 13A, another alternative embodiment of the present invention is described. Apparatus 250 comprises delivery sheath 252 that may be distally tapered to provide an atraumatic tip for advancement of apparatus 250 through a patient's body lumen. Delivery sheath 252 is translatably and coaxially disposed around catheter 254. As will be apparent to those of skill in the art, delivery sheath 252 may comprise, for example, a standard guiding catheter.

Catheter 254 of apparatus 250 comprises thermographer 256 and imaging element 170 disposed proximal of atraumatic distal tip 257. Catheter 254 further comprises catheter body 258 having guide wire lumen 260, within which guide wire 100 is illustratively disposed.

Thermographer 256 comprises a plurality of thermocouples 262 circumferentially disposed around catheter 254. Any number of thermocouples 262 may be provided. Each thermocouple 262 comprises self-expanding wire 264 proximally coupled to catheter body 258. The proximal end of each wire 264 is further electrically coupled to a processor (not shown) that captures and translates voltages generated by each thermocouple 262 into temperature values, for example, via known calibration values for each thermocouple.

Imaging element 170 comprises phased-array ultrasound transducer 172 having a plurality of discrete ultrasound elements 173 circumferentially disposed about catheter body 258 proximal of atraumatic distal tip 257. Imaging element 170 optionally may comprise multiplexing circuitry, flexible circuitry or substrates, amplifiers, etc., per se known, which may be disposed on and/or electrically coupled to apparatus 250. Transducer array 172 of imaging element 170 is electrically coupled to an imaging system (not shown), per se known, that provides excitation waveforms to the transducer array, and interprets and displays data received from the array. The imaging system coupled to imaging element 170 and the processor coupled thermographer 256 are preferably combined into a single data acquisition and analysis system (not shown) for capturing and interpreting data received from apparatus 250.

Each wire 264 is proximally affixed to catheter body 258 and is distally unfettered so that apparatus 250 may expand from the collapsed delivery configuration of FIG. 13A to the expanded deployed configuration of FIG. 13B. More specifically, when delivery sheath 252 is proximally retracted relative to catheter 254 (or catheter 254 is distally advanced with respect to delivery sheath 252), thermocouples 262 radially self-expand away from distal tip 257 to contact the interior wall of a patient's body lumen, remaining in the field of view of imaging element 170. In order to provide visual guidance during positioning of apparatus 250 at a stenosed region within the patient's body lumen in the delivery configuration, distal tip 257 and imaging element 170 of catheter 254 may be disposed partially protruding from the distal end of delivery sheath 252.

Alternatively, wires 264 may be configured so that, in the deployed configuration, thermocouples 256 contact the interior wall of the patient's body lumen immediately adjacent to the field of view of imaging element 170. This permits thermographer 256 and imaging element 170 to simultaneously obtain both temperature and imaging data at substantially the same axial position within the patient's body lumen without incorporating imaging artifacts within the acquired images.

Of course, it will be evident to one of ordinary skill in the art that the catheter embodiments of FIGS. 6 and 9-13 also may be provided as rapid exchange type catheters similar in configuration to that of FIGS. 2, 7 and 8. Specifically, rather than having guide wire lumens that span the entire longitudinal length of the catheter, the catheters of embodiments of the present invention may comprise a guide wire lumen, such as guide wire lumen 194 of FIG. 7, that proximally terminates at a side port disposed on a lateral face of the catheter. This permits a medical practitioner to rapidly exchange the catheters of the present invention with other therapeutic or diagnostic catheters.

With reference to FIG. 14, a method of using apparatus of the present invention is provided, illustratively using apparatus 180 described hereinabove. In FIG. 14, vessel V is afflicted with eccentric vulnerable plaque P that manifests only mild stenosis within vessel V. Catheter 182 of apparatus 180 is percutaneously advanced into vessel V, for example, over guide wire 100, such that imaging element 184 and thermographer 186 are disposed distally of distal edge x₀ of vulnerable plaque P, as seen in FIG. 14A. Drive cable 188 is rotated via its driver (not shown) such that imaging element 184 and thermographer 186 are provided with a full 360° view.

Catheter 182 is then withdrawn proximally across the stenosis until imaging element 184 and thermographer 186 are disposed proximally of proximal edge x₂ of vulnerable plaque P, as seen in FIG. 14B. Imaging and thermography data are collected via imaging element 184 and thermographer 186, respectively, during proximal retraction of catheter body 182 across the stenosis. Proximal retraction may be achieved manually or using a pullback system. Pullback systems are described, for example, in U.S. Pat. No. 6,290,675 to Vujanic et al., U.S. Pat. No. 6,275,724 to Dickinson et al., U.S. Pat. No. 6,193,736 to Webler et al., and PCT Publication WO 99/12474, all of which are incorporated herein by reference.

As will be apparent to those of skill in the art, catheter 182 alternatively may be advanced distally across vulnerable plaque P during data acquisition, or catheter 182 may be held stationary at a location of interest, for example, location x₁ in the middle of vulnerable plaque P. Additionally, when vulnerable plaque P has been identified, apparatus 180 optionally may be provided with stabilization elements capable of compressing, rupturing, sealing, scaffolding and/or otherwise treating the plaque in the controlled environment of a catheterization laboratory. Exemplary stabilization elements include balloon 204 of apparatus 201, and therapeutic ultrasound transducer 214 of apparatus 210. Additional stabilization elements will be apparent to those of skill in the art.

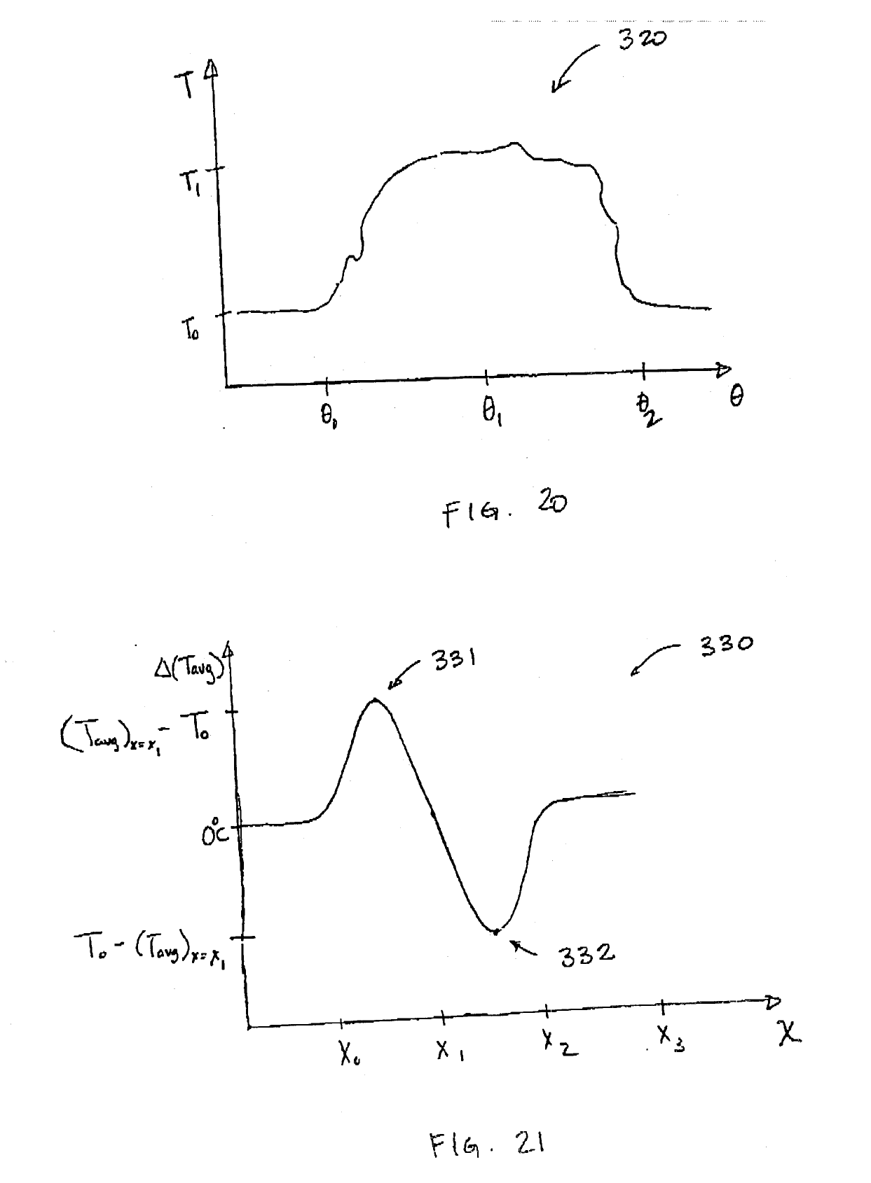

With reference now to FIG. 15, in conjunction with FIG. 14, graphical user interfaces for displaying and interpreting imaging and thermography data, collected, for example, using the methods of FIG. 14, are described. FIG. 15A provides cross-sectional IVUS image 280 formed from imaging data obtained at location x₁ within the patient's vessel V. Image 280 is eccentric and comprises echolucent zone E, which is indicative of a shallow lipid pool. Both the eccentricity and echogenicity of image 280 are indicative of vulnerable plaque P, with increased risk of rupture, at location x₁ within vessel V.

FIG. 15B displays temperature measurements T as a function of position x. Graphing temperature as a function of position requires that the position of the thermographer be recorded. Such position indication may be achieved, for example, using a pullback system, such as those described hereinabove.

In FIG. 15B, temperature measurements are obtained and graphed along angular position Y of section line A-A in FIG. 15A during proximal retraction of catheter 182 within vessel V from distal edge x₀ to location x₁ to proximal edge x₂ of vulnerable plaque P. The reference temperature within vessel V at locations proximal and distal of vulnerable plaque P is approximately T₀. All temperatures may be provided on an absolute scale, as in FIG. 15B, or temperatures may be provided as a relative change in temperature with respect to reference temperature T₀. Alternatively, an ambient reference temperature within the vessel may be obtained, for example, via thermosensor 161 of apparatus 150 of FIG. 6A, and all temperatures may be provided as a relative change with respect to the measured ambient temperature.

As seen in graph 282, as catheter 182 is proximally retracted across vulnerable plaque P, the temperature at the interior wall of vessel V along point Y rises from reference temperature T₀ to local maximum temperature T₁. Temperature T₁ is obtained at location x₁ within vessel V. The temperature within the vessel recedes back to reference temperature T₀ while catheter body 182 is further retracted from location x₁ to proximal edge x₂ of vulnerable plaque P. The increase in temperature from reference temperature T₀ to temperature T_{1 in the region surrounding location x}₁ within the vessel may be as much as about 0.10° C. to over 2.0° C., and is typically at least 0.3° C. This range is provided only for the purpose of illustration and should in no way be construed as limiting.

The increase in temperature from T₀ to T₁ is indicative of vulnerable plaque susceptible to rupture. By comparing and correlating the thermographic data of graph 282 of FIG. 15B to IVUS image 280 of FIG. 15A, identification of vulnerable plaque P is corroborated and confirmed. Thus, providing both imaging and thermography simplifies vulnerable plaque identification while reducing a level of skill required on the part of a medical practitioner in order to properly diagnose such plaque.

In addition to graphing temperature measurements as a function of position, temperature measurements alternatively may be displayed as dynamic, individual measurements (not shown) obtained at the current position of the thermographer. As yet another alternative, temperature measurements may be displayed for an entire vessel cross-section (see FIG. 16), such as a cross-section of temperature measurements obtained at location x₁. Cross-sections of thermography and imaging data at a given position may be compared to provide rapid and proper identification of vulnerable plaque.

Referring now to FIG. 16, a graphical user interface for concurrently displaying both imaging and thermography data is described. In FIG. 16, imaging and thermography data are correlated and coupled prior to display, for example, using position indication techniques and/or a pullback system, such as an IVUS pullback system that is modified to simultaneously monitor the position of both the imaging element and the thermographer. Determination of the distance between imaging elements and thermographers on integrated catheters of the present invention is also expected to facilitate coupling. Optional stablization elements also may be monitored via position indication techniques and/or a pullback system. IVUS pullback systems are described hereinabove.

In FIG. 16, imaging and thermography data, are simultaneously displayed on separate scales in a graphical, overlaid fashion, for example, on a standard computer monitor. Graphical user interface 290 comprises imaging cross-section 292 and thermography cross-section 294. Both imaging cross-section 292 and thermography cross-section 294 were obtained at location x₁ within vessel V. Imaging cross-section 292 is eccentric and contains echolucent zone E, which is indicative of a shallow lipid pool.

Thermography cross-section 294 is displayed with reference to temperature intensity scale S that ranges between T₀ and T_{1. Scale S may be provided as a color shift, an intensity shift, or a combination thereof. Furthermore the line width along thermography cross-section 294 may be altered to indicate changes in temperature. Additionally, the range of scale S may be extended beyond T}₀ and T₁, or may be displayed as a change in temperature ΔT from a reference background temperature, such as T₀. Additional scales S will be apparent to those of skill in the art and are included in the present invention. As can be seen in FIG. 16, the intensity of thermography cross-section 294, and thus the temperature within vessel V, increases along eccentric echolucent zone E of imaging cross-section 292, which is indicative of vulnerable plaque.

Overlaying imaging and thermography data on separate scales facilitates rapid correlation of the temperature at a given position within vessel V to the image obtained at that position. Rapid correlation is expected to simplify, expedite and increase the accuracy of vulnerable plaque identification. As will be apparent to those skilled in the art, as an alternative to providing temperature and imaging data on separate scales within the same graphical user interface, the imaging data may be color-coded (not shown) to indicate temperature. Additional data may also be obtained, coupled and provided in the graphical display, for example, elastography or palpography data (not shown). Palpographic techniques are described, for example, in U.S. Pat. No. 6,165,128 to Cespedes et al., which is incorporated herein by reference. Blood flow imaging may also be provided (not shown). Blood flow imaging is described, for example, in U.S. Pat. Nos. 5,453,575 and 5,921,931 to O'Donnell et al., both of which are incorporated herein by reference.

Referring now to FIG. 17, an alternative graphical user interface that simultaneously displays coupled imaging and thermography data is described. Graphical user interface 300 overlays imaging and thermography data in a manner similar to interface 290 of FIG. 16. However, interface 300 displays data obtained along side-sectional view line B-B of FIG. 16 during retraction or advancement of apparatus of the present invention across vulnerable plaque P. Retraction or advancement across plaque P is preferably achieved using a modified IVUS pullback system, as described hereinabove.

Graphical user interface 300 comprises imaging side-section 302 and thermography side-section 304. Imaging side-section 302 is eccentric and comprises echolucent zone E, which is most pronounced in the region around location x₁ within vessel V. Likewise, thermography side-section 304 is of greatest intensity in the region around echolucent zone E of imaging side-section 302. Concurrent analysis of imaging side-section 302 and correlated thermography side-section 304 is expected to facilitate improved identification of vulnerable plaque. As with the cross-sectional view of graphical user interface 290 of FIG. 16, image side-section 302 may alternatively be color-coded to indicate temperature (not shown). Furthermore, additional information, for example, palpography information or blood flow information, may be provided within the side-sectional view of graphical user interface 300, in order to further facilitate plaque identification. The additional data, e.g. the palpography data or the blood flow data, is preferably obtained concurrently with imaging data, for example, via the imaging element.

As will be apparent to those of skill in the art, as an alternative to presenting imaging and thermographic data as side-sections and/or cross-sections, such data may be provided as partial or complete 3-dimensional reconstructions (not shown).

In accordance with another aspect of the present invention, temperature measurements (as well as imaging intensity or echogenicity, etc.) alternatively may be displayed on a 3-dimensional graph as a function of both axial vessel position and angular position. For example, FIG. 19 illustratively provides 3-dimensional graph 310 having coordinate axes that correspond to temperature T, axial position x and angular position θ. Graph 310 illustratively provides temperature data that may be obtained by any of the embodiments of the present invention, for example, by catheter 182 of FIG. 14 when catheter 182 is retracted and rotated in the manner described above within vessel V of FIG. 18. In particular, graph 310 provides illustrative temperature measurements along the vessel wall as a function of axial position x and angular position θ, approximately bounded by an area coincident with vulnerable plaque P. This area approximately is limited within the angular measurements θ₀ to θ₂, and axial positions x₀ to x₂. Clearly, an entire 360° angular view alternatively may be provided. The reference temperature within vessel V at locations peripheral to and outside of this area is approximately T₀. All temperatures may be provided as a relative change in temperature with respect to reference temperature T₀, or temperatures may be provided on an absolute scale, as in FIG. 19.

As seen in graph 310, as catheter 182 is rotated and/or retracted across vulnerable plaque P, the temperature at the interior wall of vessel V increases from reference temperature T₀ to local maximum temperature T₁. The temperature within vessel V recedes back to reference temperature T₀ as catheter 182 is rotated and/or retracted past vulnerable plaque P.

In accordance with another aspect of the present invention, graph 310 may be interactive, allowing a medical practitioner to examine areas of interest, such as a local maximum or minimum, in greater detail by selecting indicia along the coordinate axes. For example, if angular position θ₁ is selected, a graphical user interface then may provide a 2-dimensional graph, such as graph 282 of FIG. 15B, of temperature measurements along the vessel wall at angular position θ₁. Alternatively, selection of angular position θ₁ may provide a side-sectional view of vessel V with thermography data overlaid thereon, such as graphical user interface 300 of FIG. 17.

Likewise, upon selection of a specific axial position, a 2-dimensional graph of temperature along the vessel wall as a function of angular position θ may be provided at that specific axial position. For example, if axial position x₁ is selected on graph 310 of FIG. 19, graph 320 of FIG. 20 may be provided. As may be seen from graph 320, the temperature at the vessel wall at angular positions less than 74 ₀ and greater than θ₂ approximately equal reference temperature T₀, whereas the temperature at angular positions between θ₀ and θ₂ are approximately equivalent to local maximum temperature T₁. The higher temperature of the vessel between θ₀ and θ₂ is indicative of the presence of vulnerable plaque P with an increased risk of rupture. Alternatively, instead of graph 320, selection of axial position x₁ may display a cross-sectional view of vessel V at axial position x₁ with the temperature data overlaid thereon, as illustrated in graphical user interface 290 of FIG. 16.

The user also may elect to obtain more detailed information about a specific temperature value. For example, selection of temperature T₁ on graph 310 of FIG. 19 would provide a 2-dimensional graph, chart or table of the angular positions θ and axial positions x at which the temperature measured at the vessel wall equaled temperature T₁. The apparatus of the present invention then may be advanced to those identified positions for additional investigation.

Of course, one of ordinary skill in the art will recognize that, while the graphs and graphical user interfaces of FIGS. 15-20 display temperature measurements, other vessel parameters VP also may be displayed without departing from the present invention. As discussed previously, stiffness, strain and elasticity information may be obtained from elastography or palpography measurements. These parameters, along with blood flow imaging, pressure, pH and flow velocity, also may be displayed individually or simultaneously with combinations thereof. If these parameters are simultaneously displayed, the different datasets may be displayed in an overlaid fashion or as independent datasets. These vessel parameters are provided for illustrative purposes only and should in no way be construed as limiting.

In accordance with yet another aspect of the present invention, measurements of vessel parameter VP (e.g., temperature, strain, pressure and pH) may be provided as an average summation value along a cross-section or side-section of vessel V. Average summation values may be used in rapid bulk testing to narrow the region(s) within vessel V that require additional analysis. Mathematically, the average summation of vessel parameter VP may be computed, for example, as follows:

$${VP}_{avg} = ( {\sum }_{i = 1}^{i = n} {VP}_{i} ) / n \qquad EQ . 1$$

wherein VP is the vessel parameter of interest, such as temperature; n is the number of VP measurements taken along a given region of interest, such as a side-section or cross-section of vessel V; and i is the specific measurement of VP being examined.

As one of ordinary skill in the art will recognize, n will depend on the frequency of data acquisition, the number of imaging transducers or elements within an imaging transducer, the number of thermographers, etc., disposed within the apparatus of the present invention.

The value VP_{avg} may be displayed in a variety of ways, such as a numerical display, a color/intensity coded value in which the color/intensity is representative of the magnitude of the value and/or as an audio frequency in which the frequency increases with increasing magnitude of the value.

When VP_{avg} is calculated for multiple cross-sections or side-sections, a 2-dimensional graph may be presented in which the multiple VP_{avg} values are respectively displayed as a function of axial or angular position within vessel V.

To further facilitate rapid bulk testing, a number of methods may be used to accentuate atypical shifts or deviations in VP_{avg} values, which may be indicative of the presence of vulnerable plaque susceptible to rupture. A first method comprises raising each individual measurement of vessel parameter VP to a power, e.g., squared. The resultant average summation value may be calculated as follows:

$${VP}_{shift indictor avg} = ( {\sum }_{i = 1}^{n} {( {VP}_{i} )}^{2} ) / n \qquad EQ . 2$$

Alternatively, shifts in VP_{avg} values may be accentuated by multiplying each individual measurement of vessel parameter VP by a scaling factor C:

$${VP}_{scaled avg} = ( {\sum }_{i = 1}^{i = n} C ( {VP}_{i} ) ) / n \qquad EQ . 3$$

Yet another alternative method to accentuate shifts in VP_{avg} values subtracts out a normal value VPₙₒᵣₘₐₗ from each individual measurement of vessel parameter VP as follows:

$${VP}_{normalized avg} = ( {\sum }_{i = 1}^{i = n} ( {VP}_{i} - {VP}_{normal} ) ) / n \qquad EQ . 4$$

An illustrative value for VPₙₒᵣₘₐₗ may comprise a reference value of vessel parameter VP, such as T₀ for temperature. When VP_{normalized}_{_{—}}_{avg} is greater or less than zero, the cross-section or side-section corresponding to that VP_{normalized avg} value may require additional examination.

Shifts in VP_{avg} may be further accentuated by raising the difference between each individual value of vessel parameter VP and VPₙₒᵣₘₐₗ to a power, e.g., squared, as follows:

$${VP}_{normalized shift indicator avg} = ( {\sum }_{i = 1}^{i = n} {( {VP}_{i} - {VP}_{normal} )}^{2} ) / n \qquad EQ . 5$$

An alternative method to further accentuate shifts in VP_{avg} comprises multiplying the difference between each individual value of vessel parameter VP and VPₙₒᵣₘₐₗ by scaling factor C as follows:

$${VP}_{normalized scaled avg} = ( {\sum C}_{i = 1}^{i = n} ( {VP}_{i} - {VP}_{normal} ) ) / n \qquad EQ . 6$$

As discussed with reference to EQ. 1, average summation values calculated using EQS. 2-6 may be provided as a numerical display, a color/intensity coded value, or an audio frequency.

It also may be desirable to examine vessel parameter VP in a third dimension. Gradients may be calculated to detect rapid changes in the average summation values VP_{avg} between successive cross-sections or side-sections of vessel V. Large gradients may be indicative of areas within vessel V that require additional examination or the presence of vulnerable plaque P susceptible to rupture. To determine the change in average summation values VP_{avg} between successive cross-sections or side-sections of vessel V, the following calculation may be made:

∇(VP_{avg})=VP_{avg,p+1}−VP_{avg,p} EQ. 7

wherein p, the specific measurement of VP_{avg} being examined, ranges from 1 to m, wherein m is the number of cross-sections or side-sections for which VP_{avg} has been calculated along the length or angular section of vessel V that is of interest.

To display the gradients computed with EQ. 7, ∇(VP_{avg}) may be graphed as a function of axial position x if values of ∇(VP_{avg}) are calculated for successive cross-sections of vessel V, or as a function of angular position θ if values of ∇(VP_{avg}) are calculated for successive side-sections of vessel V.

Graph 330 of FIG. 21 illustrates EQ. 7, wherein temperature T is used as vessel parameter VP. Axial positions x₀−x₃ correspond to the same axial positions denoted in FIG. 18. Specifically, axial positions x₀ and x₂ respectively represent the distal and proximal ends of vulnerable plaque P, x₁ represents an axial location in the middle of vulnerable plaque P, and x₃ represents an axial position proximal to vulnerable plaque P. As discussed previously, the temperature at axial positions x₀, x₂ and x₃ are approximately equal to reference temperature T₀, whereas the temperature at axial position x₁ approximately equals elevated temperature T₁. Accordingly, T_{avg} of the cross-sections of vessel V that correspond to axial positions xₒ, x₂ and X₃ would equal T₀, while T_{avg} of the cross-section at axial position x₁ (i.e., (T_{avg})ₓ₌ₓ₁) would be greater than T₀. When EQ. 7 is applied to each axial position, illustrative results of which are shown on graph 330 of FIG. 21, gradient shifts 331 and 332 are noticeable between axial positions x₀ and x₂. In addition to visual confirmation from images provided by imaging element 184, shifts 331 and 332 may be indicative and may provide notice of the presence of vulnerable plaque P in vessel V with increased risk of rupture.

As in EQ. 1, an average gradient value for ∇(VP_{avg}) may be calculated for the length or angle of interest as follows:

$$∇ {( {VP}_{avg} )}_{avg} = ( {\sum }_{p = 1}^{p = m} ( {VP}_{avg , p + 1} - {VP}_{avg , p} ) ) / m \qquad EQ . 8$$

Furthermore, as in EQS. 2 and 5, shifts in gradients ∇(VP_{avg}), such as shifts 331 and 332 of FIG. 21, may be accentuated by raising each gradient to a power, e.g., squared, as follows:

∇(VP_{avg})_{shift indicator}=(VP_{avg, p+1}−VP_{avg, p})² EQ. 9

Likewise, as in EQS. 3 and 6, shifts in gradients ∇(VP_{avg}) also may be accentuated by multiplying each gradient by scaling factor C as follows:

∇(VP_{avg})_{scaled}=C(VP_{avg, p+1}−VP_{avg, p}) EQ. 10

As discussed in reference to EQ. 7, the gradients calculated by EQS. 9 and 10 may be displayed on a 2-dimensional graph as a function of axial position x or angular position θ.

Of course, one of ordinary skill in the art will recognize that ∇(VP_{avg}) shift indicator of EQ. 9 and ∇(VP_{avg})_{scaled} of EQ. 10 may be averaged over a length or angle of vessel segment that is of interest to facilitate rapid determination of whether that vessel segment requires further examination. To calculate ∇(VP_{avg})_{shift indicator avg} or ∇(VP_{avg})_{scaled avg}, EQ. 8 may be used in which ∇(VP_{avg}) is replaced with ∇(VP_{avg})_{shift indicator} or ∇(VP_{avg})_{scaled}, respectively.

It is also noted that the equations given above may be modified for use with individual measurements of vessel parameter VP. Specifically, to accentuate shifts in measurements of vessel parameter VP, and thereby facilitate rapid bulk testing, each measurement value may be raised to a power (e.g., squared), multiplied by scaling factor C, added to normal value −VPₙₒᵣₘₐₗ, or modified by combinations thereof as follows:

$$\begin{matrix}{VP}_{shift indicator} = {VP}^{2} & EQ . 11 \\ {VP}_{normalized} = VP - {VP}_{normal} & EQ . 12 \\ {VP}_{normalized shift indicator} = {( VP - {VP}_{normal} )}^{2} & EQ . 13 \\ {VP}_{scaled} = C ( VP ) & EQ . 14\end{matrix}$$

The resultant modified vessel parameter may be displayed as a numerical display, a color/intensity coded value, and/or an audio frequency.

Gradients also may be calculated for a particular axial or angular section of interest by calculating the difference in successive values obtained for vessel parameter VP, as follows

∇VP=VP_{q+1}−VP_{q} EQ. 15

wherein q ranges from 1 to s, s being the number of measurements of vessel parameter VP that have been obtained at a particular axial or angular section of vessel V that is of interest. Furthermore, shifts in gradient values calculated using EQ. 15 may be accentuated to facilitate rapid bulk testing by using EQS. 11 and 14, wherein vessel parameter VP may be replaced by ∇VP. These gradients may be displayed in a 2-dimensional graph as a function of axial position x or angular position θ.

Furthermore, rapid bulk testing may further be facilitated if average summation values are provided for the- above described gradients. Specifically, the following calculations may be made and displayed as a numerical display, a color/intensity coded value, or a radio frequency:

$$\begin{matrix}{( ∇ VP )}_{avg} = ( {\sum }_{q = 1}^{s} ( {VP}_{q + 1} - {VP}_{q} ) ) / s & EQ . 16 \\ {( ∇ VP )}_{shift indicator avg} = ( {\sum }_{q = 1}^{s} {( {VP}_{q + 1} - {VP}_{q} )}^{2} ) / s & EQ . 17 \\ {( ∇ VP )}_{scaled avg} = ( {\sum }_{q = 1}^{s} C ( {VP}_{q + 1} - {VP}_{q} ) ) / s & EQ . 18\end{matrix}$$

It will be obvious to one of ordinary skill in the art that the above discussed values also may be determined as a function of radial dimension r. Likewise, the equations also may be applied to spherical and Cartesian coordinates, as well as any other coordinate system.

Imaging through blood is a complex function of absorption and scattering or diffraction. As water is its dominant component, absorption behavior in blood is somewhat similar to that in water. Images with excessive absorption appear ‘dark’, as if greater illumination (power) is required.

Excessive absorption can typically be overcome by increasing power, changing illumination wavelength and/or changing media. However, if power is increased, substantial heat may be generated. Thus, at high powers the light source may need to be pulsed to reduce heat generation/energy transfer to the media.

When wavelength is altered, absorption tends to increase with wavelength. However, significant localized absorption minima and maxima appear due to molecular resonance, etc. It is preferable to image near absorption minima, thereby reducing required power.

Absorption in blood may also be overcome by changing the media, e.g. an alternative media may be injected, such as saline. Alternatively, blood flow may be blocked (e.g. with a balloon). However, it is important to ensure that ischemia doesn't develop.

In contrast to absorption, scattering cannot be mitigated by increasing power. Images with excessive scattering appear blurry and unfocused. As a generalization, scattering decreases as wavelength increases (i.e. as the particles—in this case blood cells—become small relative to the wavelength of the light). In part, scattering results from a change in index of refraction between a media and particles in that media; injection of alternative media or blockage of flow may dilute the concentration of particles (i.e. blood cells), thereby decreasing scattering. If alternative media is injected, it should preferably closely match the index of refraction of the blood cells, which have an index of refraction of about 1.29. Plasma has an index of refraction of about 1.35.

U.S. Pat. No. 6,178,346 to Amundson et al., incorporated herein by reference, describes scattering and absorption phenomena in significant detail. That reference outlines a few wavelength regions where an optimal balance of absorption and scattering may be obtained. It defines near infrared (“IR”) wavelengths as 800-1400 nm, mid-IR wavelengths as 1500-6000 nm, and far-IR wavelengths as 6000 to 15000 nm. Optimal properties are found at 1500-1800 nm, 2100-2400 nm, 3700-4300 nm, 4600-5400 nm, and 7000-14000 nm. U.S. patent application publication 2001/0047137 to Moreno et al., incorporated herein by reference, found optimal properties at 1450-1950 nm, and even more preferably at 1600-1800 nm. Higher wavelength techniques theoretically can visualize greater distances; however, they require significantly more power (and thereby have a significantly higher potential for heat generation) to overcome increased absorption with increased wavelength. Thus, for intravascular use, the Amundson patent recommends the 1500-1800 nm and the 2100-2400 nm ranges, and even more preferably about 1600-1700 nm or 2100-2200 nm.

US2001/0047137, to Moreno et al. also describes various light sources that may be used. Preferred light sources are wavelength tunable, which may be achieved, for example, with a filter, a monochromator (e.g. a 1000W tungsten-halogen lamp), an interferometer, or a laser (e.g. an Nd:YAG laser). One or more detectors may be provided for detecting back scattered and reflected light. A single detector is sufficient for spectrometry. A detector array is needed for imaging, and has been achieved with an Indium Antinomide focal plane array video camera. A CMOS or CCD sensor may also/alternatively be provided. The detector(s) may be coupled to an Analog/Digital converter, and an image analysis system, such as a computer with a video display. Imaging and/or data may also be recorded.

US2001/0047137 further describes the use of infrared imaging for both spatial and chemical analysis. Chemical analysis is based on a comparison of detected light with reference absorption curves for various compounds. Potential compounds for analysis include lipoproteins (including high-density lipoproteins “HDL” and low-density lipoproteins “LDL”, as well as 128 KD lypoprotein in necrotic plaques), Group V Secretory Phospholipase 2 “sPLA2”, lysophosphatidylcholine “LPC”, serum amyloid A “SAA”, cholesterol esters and cholesterol monohydrate. These compounds may indicate the presence and/or progression of plaque, including vulnerable plaque. Chemical analysis via infrared imaging may help determine a course of treatment, including, for example, lipid lowering with statins, modulation of matrix metalloproteinases “MMPs” (e.g. via specific tissue inhibitors of metalloproteinases “TIMPS”, via non-specific inhibitors such as 2-macroglobulin, via synthetic inhibitors such as those produced by Agouron Inc., or via gene therapy), and/or inhibition of sPLA2.

As discussed previously, vulnerable plaques typically exhibit a thin fibrous cap with a large lipid/atheromatous core, and macrophage infiltration. Both imaging and therapy may be achieved with an IR source, as described in US2001/0047137. Specifically, therapy may be achieved by illuminating at a sufficient power to cause calcification of the fibrous cap. For example, when using an Nd:YAG laser source, short pulses of less than about 10 ms may be provided at a power of about 100 mJ to achieve calcification.

US2001/0047137 also discusses normalization of an IR spectrum to reduce the effects of variation in water content. As for transmission of light from the light source to the media/tissue, and receipt of backscattered light from the media/tissue, fiber optic cable(s) may be provided. US2001/0047137 describes separate fiber optics for transmission and receipt. U.S. Pat. No. 6,178,346 describes the use of a beam splitter so that transmission and receipt may be achieved with the same fiber(s), thereby potentially reducing the crossing profile of catheters having an IR/light-based probe. Additionally, various optics may be provided at the distal end of the fibers to enhance, focus, redirect, etc., signal transmission and receipt. Optics arrangements are shown, for example, in U.S. Pat. No. 6,178,346 (See FIGS. 11B and 12B), US2001/0047137 (See FIGS. 13-15), as well as U.S. patent application publication 2002/0068853 to Adler (See FIGS. 2 and 4), U.S. Pat. No. 6,445,939 to Swanson et al. (See FIGS. 2 and 4-12), U.S. Pat. No. 6,134,003 to Tearney et al. (See FIGS. 6, 7 and 10-12) and U.S. Pat. No. 6,010,449 to Selmon et al, all of which are incorporated herein by reference in their entirety.

Infrared imaging involves illumination of a target site with IR light, and measurement of backscattered/reflected light to construct an image. Conversely, infrared thermography measures naturally-emitted radiation from the target site, and constructs an image/measures temperature based on the naturally-emitted radiation. Infrared thermography does not require an illuminating light source. Radiation from body tissue typically occurs in the mid- to far-IR spectrum, from about 1500-15000 nm. There is a need in the art for an intravascular device capable of both infrared imaging and infrared thermography.

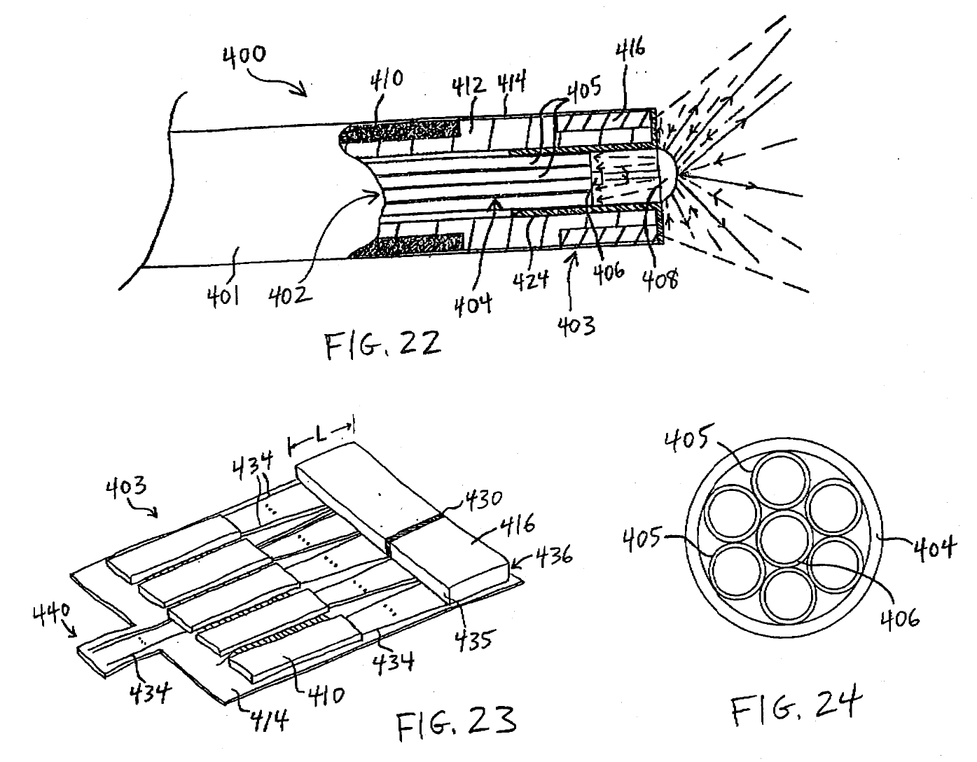

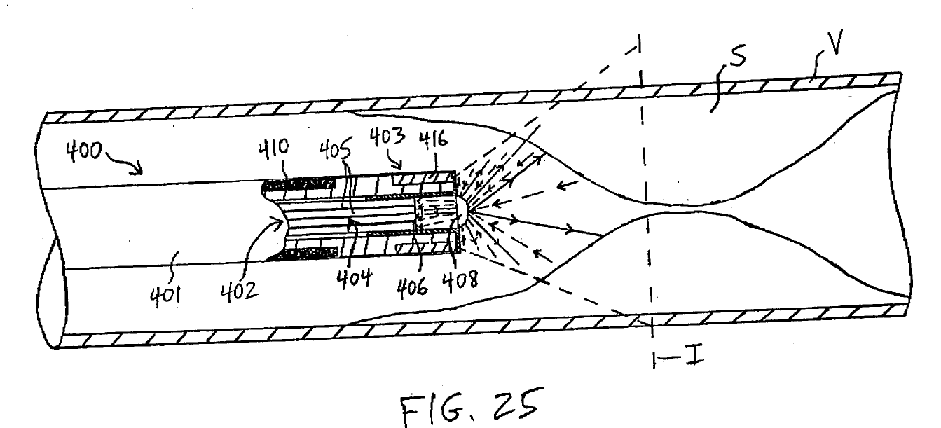

Referring now to FIGS. 22-25, a further alternative embodiment of the present invention is described that provides both an imaging element and an infrared element in a single device. By providing both imaging and infrared elements in a single device, the present invention combines advantages associated with stand-alone imaging and infrared devices into a single device. In particular, an image map may be constructed using, e.g., IVUS, as described in detail hereinabove, while chemical, thermographic and/or emissivity analyses of vessel characteristics may be performed using infrared techniques. Therefore, a medical practitioner may identify vulnerable plaque using IVUS, and then use the infrared element to provide a secondary indication or confirmation of vulnerable plaque via a secondary analysis of the vessel. Furthermore, therapy may be achieved using the infrared source, e.g., by illuminating a region of vulnerable plaque at a power sufficient to cause calcification of the fibrous cap of the vulnerable plaque. Accordingly, apparatus 400 of the present invention may serve as an imaging tool, a chemical, thermographic and/or emissivity analysis tool, and a vulnerable plaque treatment or stabilization tool, all in one.

Referring now to FIG. 22, apparatus 400 of the present invention comprises catheter body 402, IVUS imaging assembly 403, and infrared analysis assembly 404. IVUS imaging assembly 403 is disposed at a distal region of catheter body 402 and preferably is forward-looking, as described, for example, in U.S. Pat. No. 6,457,365 to Stephens et al., which is hereby incorporated by reference in its entirety.

In particular, IVUS imaging assembly 403 comprises plurality of transducer elements 416 that are arranged in a cylindrical array centered about a longidutinal axis of catheter body 402 for transmitting and receiving ultrasonic energy. Transducer elements 416 are mounted on an inner wall of substrate 414 that comprises, for example, a flexible circuit material that has been rolled in the form of a tube. A transducer backing material 412 having proper acoustical properties surrounds transducer elements 416. End cap 424, which covers a distal end of transducer elements 416, may be used to insulate the transducer elements from external fluid, such as blood.

Referring to FIG. 23, a preferred method of fabricating IVUS imaging element 403 is briefly described to facilitate understanding of the operation of IVUS imaging element 403 of FIG. 22. A detailed description of the preferred method of fabricating IVUS imaging element 403 is described in applicant's pending U.S. patent application Ser. No. 10/233,870, which is hereby incorporated by reference in its entirety.

In FIG. 23, transducer elements 416 may have a number of individual elements, each of which is aligned in parallel with illustrative element 430 shown in FIG. 4. Transducer elements 416 are mounted on flex circuit 414, e.g., a flexible substrate material such as polyimide, which is electrically insulating. If desired, the flex circuit may be formed from a substance having a relatively high acoustical impedance for flexible polymeric materials.

Electrical conductors 334 are formed on the surface of flex circuit 414, as shown in FIG. 23. The electrical conductors may be formed, for example, from a malleable metal such as gold or copper. A suitable adhesion layer such as a thin layer of chromium may be used to facilitate adhesion of the conductor material to the flex circuit. Metal layers may be deposited by sputtering, evaporation, or any other suitable technique. Wet or dry etching, or other suitable patterning techniques, may be used to pattern the deposited metal to form electrical conductors 34.

Each transducer element 430 may have two opposing electrodes. The main portion of the electrodes is located on the upper and lower surfaces of the transducer array when the array is oriented as shown in FIG. 23. Smaller portions of the electrodes extend over the ends 435 and 436 of the elements 430 in transducer array 416. Electrical signals may be conducted between the conductors 434 and the main portions of the electrodes by forming electrical contacts between the conductors 34 and the end portions 435 and 436.

By connecting the electrodes on each transducer element 430 to corresponding conductors 434, drive signals for the transducer elements 30 may be conveyed to the elements 430. Similarly, electrical signals that are produced by the elements 430 when reflected acoustic waves are detected by elements 430 may be conveyed from the elements.

In some transducer arrays (e.g., arrays with 64 elements or more), there may be so many conductors 434 that it is cumbersome to route all of these conductor lines to processing equipment in a single cable along the length of catheter body 402. Accordingly, integrated circuits 410 (e.g., time-division multiplexing circuits or other suitable multiplexing circuits) may be used to reduce the relatively large number of conductors 434 that are directly connected to transducer array 416 into a smaller number of conductors 434 at the input/output 440. The conductors at input/output 440 may be soldered, welded, or otherwise electrically connected to wires in a suitable cable (not shown) that runs along the length of catheter body 12 to suitable image processing equipment. If desired, integrated circuits 410 may include drive circuitry for generating drive signals and/or preprocessing circuitry for at least partially processing the electrical signals that are produced when the transducer elements 430 in array 428 are used to detect acoustical information.

After circuits 410 and transducer array 28 have been mounted on flex circuit 414, as shown in FIG. 23, flex circuit 414 and its mounted components is formed into a cylindrical shape and attached to the distal section of catheter body 402, as shown in FIG. 22.

Integrated circuits 410 and array 416 preferably are wrapped about a fiber optic bundle of infrared analysis element 404, which is described in detail hereinbelow. End cap 424 also may be disposed partially between IVUS imaging element 403 and infrared analysis element 404 to isolate ends 436 of elements 430 of array 416 from blood flow. Backing material 412, as described hereinabove, also is disposed between IVUS imaging element 403 and infrared analysis element 404, as shown in FIG. 22.

Referring to FIG. 22, infrared analysis assembly or element 404 preferably comprises a fiber optic bundle, which is disposed within IVUS imaging apparatus 403. A plurality of fiber optic strands may be disposed within the fiber optic bundle of infrared element 404 for transmitting and receiving infrared signals. Alternatively, as will be described hereinbelow, a single fiber may be used to transmit and receive signals, e.g., using a beamsplitter or timed pulses.

Referring now to FIG. 24, a cross-sectional view along a longitudinal axis of the fiber optic bundle of infrared element 404 of FIG. 22 is shown. As seen in FIG. 24, the fiber optic bundle is preferably similar to an arrangement described in patent publication No. U.S. 2001/0047137 (“the '137 publication”), incorporated by reference. The fiber optic bundle of infrared element 404 includes centrally disposed fiber optic strand 406, which is used to transmit signals, and a plurality of fiber optic strands 405 concentrically disposed about centrally disposed strand 406. A proximal end of transmitting strand 405 is coupled to a source, while each receiving strand 405 is coupled to a detector. The source and detector in turn are coupled to a processor configured to analyze the spectra detected by the detectors and produce color images of the backscattered light.

In vivo apparatus described in the '137 publication is adapted for side-viewing infrared analysis, but is not suited for in vivo forward-looking infrared analysis, as in the embodiment of FIGS. 22-25. Furthermore, in the present invention, infrared analysis is conducted in conjunction with IVUS imaging techniques described hereinabove using a single catheter. The relative positions of imaging element 402 and infrared element 404 are preferably known to facilitate correlation of imaging and infrared data.

Referring back to FIG. 22, optional optics 408, e.g. a concave lens, may be fixedly disposed at a distal end of catheter body 402. As an alternative to a concave lens, optics 408 may comprise positioning optical fibers 405 and 406 flush with a distal end of catheter body 402, and specifying their numerical aperture (“NA”) to provide a cone of light with desired angular shape, for example, between about 30° and 80°. Additional optics schemes are provided, for example, in U.S. Pat. No. 6,445,939 to Swanson et al., U.S. Pat. No. 6,178,346 to Amundson et al., U.S. Pat. No. 6,134,003 to Tearney et al., and U.S. Pat. No. 6,010,449 to Selmon et al., all of which are incorporated herein by reference. Optics 408 preferably are configured to enhance, focus and/or redirect light that is transmitted from transmitting fiber optic strand 406 to a patient's tissue. Furthermore, optics 408 preferably are configured to enhance, focus and/or redirect light that is backscattered from the tissue to receiving fiber optic strands 405.

Apparatus 400 optionally may comprise a guide wire lumen (not shown), disposed, for example, along catheter body 402 between integrated circuit 410 and the fiber optic bundle of infrared element 404. Alternatively, a small tube (not shown) may be attached to an exterior surface of catheter body 402 to serve as a guide wire lumen, e.g. a rapid exchange guide wire lumen. Additional placements and configurations for a guide wire lumen will be apparent to those skilled in the art.

Referring now to FIG. 25, a preferred method of using apparatus 400 of FIG. 22 in the detection and characterization of vascular stenosis, illustratively a total vessel occlusion, is described. In a first step, catheter body 402 of FIG. 22 is percutaneously inserted into vessel V, e.g. over a guide wire. Catheter body 402 is advanced until a distalmost region of catheter body 402 is disposed proximal of stenosis S, as shown in FIG. 25.

A processor and graphical user interface are provided for displaying and interpreting imaging and infrared data provided by apparatus 400. As described hereinabove with respect to FIG. 15A, the graphical user interface may generate a cross-sectional IVUS image similar to image 280 of FIG. 15A and/or a longitudinal or side-sectional image similar to image 300 of FIG. 17. The image provided by IVUS imaging assembly 403 may indicate the presence of a total occlusions when catheter body 402 is disposed proximal of the stenosis S. The IVUS image further may indicate echolucent zones within the total occlusion or shadowed, which are indicative of tissue-type.

In accordance with principles of the present invention, the forward-looking IVUS image generated from IVUS imaging apparatus 403 is used in conjunction with data obtained from infrared analysis assembly 404, to facilitate characterization of the vascular occlusion. Specifically, when the distal end of catheter body 402 is positioned proximal of the occlusion formed by stenosis S in vessel V, light is transmitted from a light source (not shown) that is operatively connected to transmitting fiber optic strand 406. Transmitting fiber optic strand 406 then directs the light through optional optics 408, and the light is focused and directed onto a desired region of the occlusion.

A bolus of fluid, e.g, saline, optionally may be provided to reduce scattering of the infrared light. Fluid with an index of refraction similar to blood is preferred. Alternatively, blood flow optionally may be occluded temporarily to reduce scattering.Backscattered light reflected from stenosis S then is directed into receiving fiber optic strands 405. Receiving fiber optic strands 405 direct the light to at least one detector coupled to an image analysis system.

A detector array, such as an Indium Antinomide focal plane array video camera, may be used to faciliate imaging of the backscattered and reflected light. A CMOS or CCD sensor may also be used, either alone or in combination with an array video camera. The detector array may be coupled to an analog/digital converter, which is coupled to the image analysis system, such as a computer or processor with a video display and/or recording means.

The image analysis system preferably provides a chemical analysis of the spectra detected by the detection means, based on a comparison of detected light with reference absorption curves for various compounds. These compounds may indicate the presence and/or progression of plaque, including vulnerable plaque. Potential compounds for analysis include lipoproteins (including high-density lipoproteins “HDL” and low-density lipoproteins “LDL”, as well as 128 KD lypoprotein in necrotic plaques), Group V Secretory Phospholipase 2 (“sPLA2”), lysophosphatidylcholine (“LPC”), C-reactive proteins, serum amyloid A (“SAA”), cholesterol esters and cholesterol monohydrate. Chemical analysis via infrared imaging may help determine a course of treatment, including, for example, lipid lowering with statins, lowering of C-reactive proteins, modulation of matrix metalloproteinases (“MMPs”), e.g., via specific tissue inhibitors of metalloproteinases (“TIMPS”), via non-specific inhibitors such as 2-macroglobulin, via synthetic inhibitors such as those produced by Agouron Inc., or via gene therapy), and/or inhibition of sPLA2.

In accordance with one aspect of the present invention, the infrared imaging data collected may be used in conjunction with an IVUS image to indicate the presence of the above-described compounds on an IVUS image. This is advantageous for detecting a vulnerable plaque, total occlusion, thrombus, or other stenosis and characterizing the chemical composition of the stenosis, confirming the characterization, and selecting an appropriate treatment based on the data provided by the imaging and the infrared apparatus.

Various light sources may be used in conjunction with infrared analysis apparatus 404 to transmit light to a patient's vessel. The light source preferably is adapted for generating a spectrum of light having one or more wavelengths in a range from about 800 to 14000 nm. The light source is preferably wavelength-tunable, which may be achieved, for example, using a filter, a monochromator, e.g., a 1000W tungsten-halogen lamp, an interferometer, or a laser, such as an Nd:YAG laser.

The transmission of light between the light source and a patient's vessel may be accomplished using different fiber optic strands for transmitting and receiving light, or alternatively may be accomplished using a single fiber to transmit and receive light. For example, timed pulses may be used to transmit a pulse of light on a single fiber and receive backscattered light from the pulse on the same fiber, before sending a subsequent pulse to gather additional data. Alternatively, a beamsplitter may be used to transmit and receive light using a single fiber, for example, as described in U.S. Pat. No. 6,178,346 to Amundson et al., which is incorporated herein by reference in its entirety.

Referring back to FIG. 25, apparatus 400 may further preferably comprises a means for treating total occlusion S, such as an ablation device. The means for treating may include using radiofrequency (RF) ablation by switching the frequency of the signal employed to image/chemically analyze vessel V to a signal suitable for RF ablation. Alternatively, a separate ablation device, such as a laser, RF or acoustic ablation device, or an atherectomy device, may introduced into vessel V to treat total occlusion S after apparatus 400 has been withdrawn from the vessel.

In addition, or as an alternative, to conducting chemical analysis with infrared element 404 of apparatus 400, thermography may be achieved by simply detecting naturally-emitted infrared radiation from stenosis S and/or vessel V to determine temperature without transmitting light from element 404. Blood flow is preferably temporarily occluded, e.g. with a balloon catheter, when element 404 is used as a thermographer. Furtherstill, infrared element 404 may be used to measure emmisivity of stenosis S and/or vessel V by first heating the target tissue, and then detecting naturally-emitted infrared radiation. Heating of the target tissue may be achieved, for example, by transmitting an electromagnetic frequency capable of heating from infrared element 404.

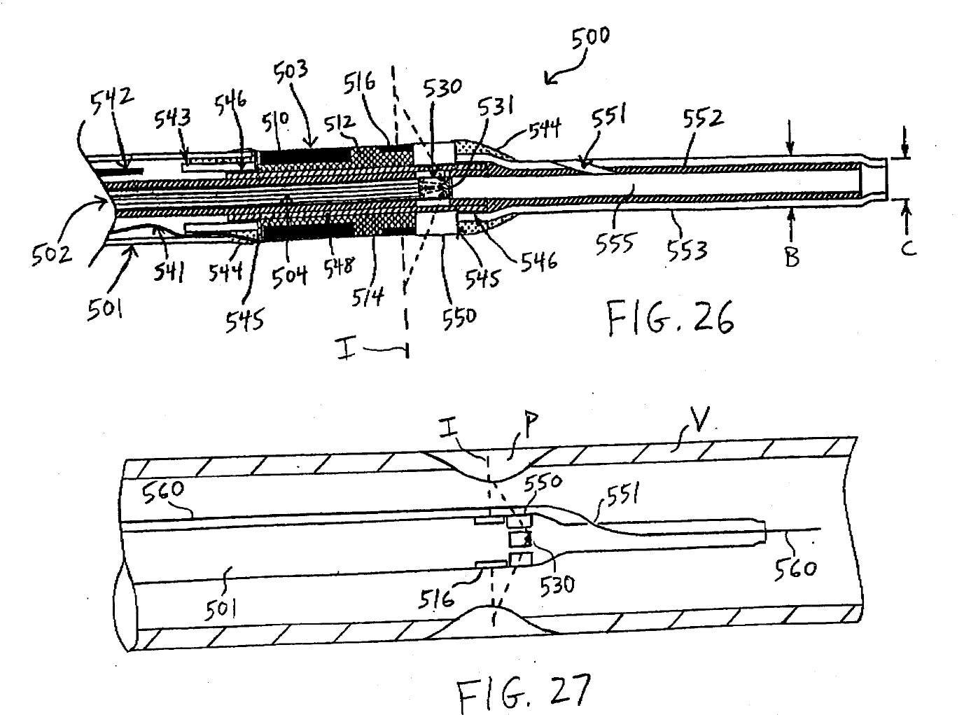

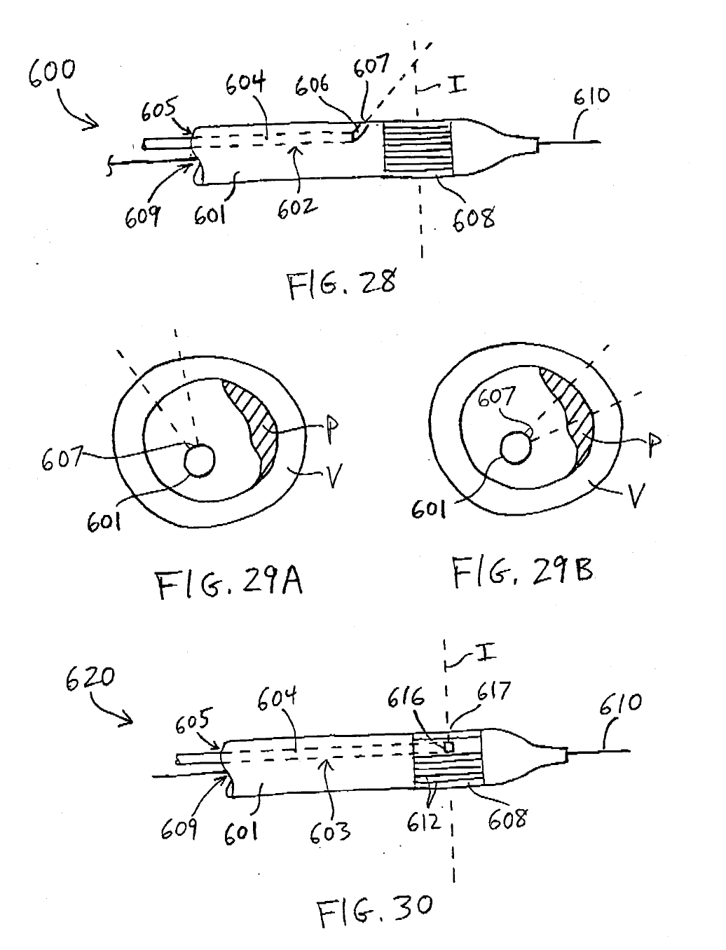

Referring now to FIG. 26, an alternative embodiment of the present invention is described for use in detecting and characterizing plaque, e.g. vulnerable plaque. Apparatus 500 of FIG. 26 comprises a catheter body 502 having side-viewing imaging apparatus 503, illustratively side-viewing IVUS imaging apparatus, as well as side-viewing infrared analysis apparatus 504.

IVUS imaging apparatus 503 preferably is provided in accordance with IVUS imaging apparatus 403 of FIGS. 22-23. Specifically, after integrated circuits 510 and transducer array 516 are mounted on flex circuit 514, as shown in FIG. 23, the flex circuit and mounted components are formed into a cylindrical shape and attached to the distal section of catheter body 502.

Catheter body 502 may have a guidewire tube 522 (e.g., a high-density polyethlyene tube) surrounded by outer tube 553, e.g., a medium-density polyethylene tube and a corresponding extension tube 543. Integrated circuits 510 and transducer array 516 may be wrapped around optically transmissive marker tube 548, e.g., comprising polycarbonate, and backing material 512.

At the input/output of flex circuit 514, cable wire 541 is connected to conductors mounted on the flex circuit, for example, using a solder or weld. Catheter 502 may have a longitudinal lumen through which cable wire 541 extends and connects to image processing and display equipment disposed proximal of the catheter body.

A distal end of catheter body 502 may be affixed to extension tube 543 using cyanoacrylate adhesive 546. Cyanoacrylate adhesive also may be used as the adhesive 546 for affixing outer tube 553 and extension tube 543 to optically transmissive marker tube 548. An ultraviolet-curable adhesive 544 may be used to seal and attach other regions of IVUS imaging apparatus 503 to the rest of catheter 502.

Additionally, optically transmissive film 550 is disposed about optically transmissive marker tube 548 and is situated between transducer array 516 and outer tube 553, as shown in FIG. 26. Optically transmissive film 550 is substantially flush with an outer surface of flex circuit 514. In a preferred embodiment, a first radiopaque marker tube washer 545 is disposed between catheter 502 and IV/US imaging assembly 503, and a second radiopaque marker tube washer 545 is disposed between optically transmissive film 550 and outer tube 553.

It will be apparent to those skilled in the art that the above-described arrangement is merely one suitable arrangement for mounting flex circuit 514 and components such as integrated circuits 510 and transducer array 516 to catheter 502. Any suitable arrangement may be used if desired. For example, separate tubes may be provided as unitary structures. Single tubes or structures may be provided in the form of individual parts that are affixed using adhesives or other suitable arrangements, and different types of tubing or adhesives may be used. Additionally, stiffening member 542 may be used to stiffen a proximal portion of catheter 502, particularly during advancement of the catheter into a patient's vessel. Furthermore, imaging elements other than IVUS imaging elements may be used, including, for example, MRI and OCT imaging elements.

In accordance with principles of the present invention, IVUS imaging assembly 503 is used in conjunction with infrared analysis assembly 504 to facilitate detection and characterization of plaque, e.g. vulnerable plaque, in a patient's vessel. Preferably, data obtained from imaging assembly 503 and infrared assembly 504 lie within the same imaging plane I.

Infrared analysis assembly 504 preferably comprises substantially cylindrical shaped housing 530, which houses reflector element 531. Reflector element 531 preferably comprises an inverted parabolic shape, as depicted in FIG. 27. Housing.530 further preferably comprises a closed distal end formed of a suitable material, such as glass. A similar infrared assembly is described in the '137 patent publication, discussed hereinabove and incorporated by reference.

Assembly 504 comprises a fiber optic bundle, which extends the length of catheter 502 and is concentrically disposed within IVUS imaging assembly 503, just proximal of reflector element 531. The fiber optic bundle preferably is provided in accordance with the fiber optic bundle described hereinabove with respect to the embodiment of FIGS. 22 and 24, so that a single transmitting fiber strand transmits lights onto reflector element 531, while a plurality of receiving strands receive backscattered light via reflector element 531, as described in detail hereinbelow with respect to FIG. 27. Alternatively, a single fiber optic strand may be used in lieu of a fiber optic bundle, in which case beamsplitting or timed pulses may be used to separate transmitting and receiving pulses. Beamsplitting techniques are described, for example, in U.S. Pat. No. 6,178,346, incorporated herein by reference.

In a preferred embodiment, outer diameter of catheter 502 and flex circuit 514 is less than about 4 French. A distal region of apparatus 500 preferably has a reduced outer diameter B of about 2.0 French, and further has a reduced diameter distal end C of about 1.8 French. Alternative dimensions will be apparent to those of skill in the art.

Referring now to FIG. 27, a preferred method of using apparatus 500 of FIG. 26 to facilitate detection and characterization of vulnerbale plaque is described. In FIG. 27, vessel V is afflicted with eccentric vulnerable plaque P that manifests only mild stenosis within vessel V. In a first step, catheter 502 is percutaneously advanced into vessel V, for example, over guide wire 560 via guide wire side port 551. Guide wire side port 551 transitions into guide wire lumen 555 to permit a medical practitioner to rapidly exchange the catheters of the present invention with other therapeutic or diagnostic catheters.

Catheter 502 of apparatus 500 is percutaneously advanced into vessel such that transducer array 516 of IVUS imaging apparatus 503 and housing 530 of infrared imaging apparatus 504 are disposed distally of a distal edge of vulnerable plaque P. Catheter 502 may be withdrawn proximally across the stenosis, e.g., manually or using a pullback system, as described hereinabove, until tranducer array 516 and housing 530 are disposed proximal of a proximal edge of vulnerable plaque P.

As catheter 502 is retracted within vessel V, transducer array 516 provides cross-sectional images of vessel V over a range of longitudinal locations within the vessel. A side view of vessel V, for example, as shown in FIG. 17 hereinabove, may be generated on a computer display using information gathered from transducer array 516.

In accordance with principles of the present invention, the side-viewing IVUS imaging data generated from IVUS imaging apparatus 503 is used in conjunction with data obtained from infrared element 504, to facilitate characterization of vulnerable plaque within a vessel. Specifically, as catheter 502 is retracted within vessel V, light is transmitted from a light source that is operatively connected to a transmitting fiber optic strand, e.g., strand 406 of FIG. 24. Transmitting fiber optic strand 406 then directs the light onto reflector element 531, which then redirects the light in a direction depicted in FIG. 27. Light is directed through optically transmissive marker tube 548, optically transmissive film 550, and onto a region of vessel V coinciding with the IVUS imaging data, such as plaque P, as shown in FIG. 27. Scattered light reflected from the region then is directed back into recieving fiber optic strands 405 of FIG. 24, which then direct the light to at least one detector coupled to an image analysis system.

As described hereinabove with respect to FIG. 25, the image analysis system provides an analysis of the spectra detected by the detection means, based on a comparison of detected light with reference absorption curves for various compounds, as described, for example with respect to patent publication US2001/0047137, incorporated herein by reference. In accordance with one aspect of the present invention, the infrared data collected may be used in conjunction with imaging to indicate the presence of above-described compounds on an image formed from the imaging data. This is advantageous for detecting and characterizing vulnerable plaque P within vessel V, so that an appropriate treatment based on the data provided by the IVUS and infrared apparatus may be selected.

As will be apparent to those skilled in the art, catheter 502 alternatively may be advanced distally across vulnerable plaque P during data acquisition, or catheter 502 may be held stationary at a location of interest, for example, in the middle of plaque P, e.g. vulnerable plaque. Additionally, when vulnerable plaque P has been identified, apparatus 500 optionally may be provided with stabilization elements capable of compressing, rupturing, sealing, scaffolding and/or otherwise treating the plaque in the controlled environment of a catheterization laboratory. Exemplary stabilization elements include balloon 204 of apparatus 201, and therapeutic ultrasound transducer 214 of apparatus 210. Additional stabilization elements will be apparent to those of skill in the art.

As with the previous embodiment, infrared analysis may be enhanced by using a bolus of fluid to reduce scattering of light by blood, or flow may temporarily be blocked.

Referring now to FIG. 28, an alternative embodiment of the device of FIGS. 26-27 is described for use in detecting and characterizing vulnerable plaque using an IVUS imaging element in conjunction with an infrared imaging/analysis element. In FIG. 28, apparatus 600 comprises catheter 602 having infrared imaging element 603 and IVUS imaging element 608. IVUS imaging element 608 preferably comprises a side-viewing array of transcuder, as described in detail with respect to IVUS imaging element 503 of FIG. 26 hereinabove.

Infrared imaging element preferably comprises fiber optic 604. Fiber optic 604 may include distinct transmitting and receiving strands, for example, as described hereinabove with respect to FIG. 24, or alternatively may comprise a single strand that uses beamsplitting or timed pulses to transmit and receive light.

Fiber optic 604 extends through lumen 605 of catheter 602, which terminates distally at side port 607. A proximal end of fiber optic 604 is coupled to a transmitting light source, and further coupled to backscattered light detectors and image display and processing apparatus, as described hereinabove with respect to the embodiment of FIGS. 22-25. A distal end of fiber optic 604 transmits light, optionally via optics, through side port 607 and onto a region of interest in a patient's vessel.

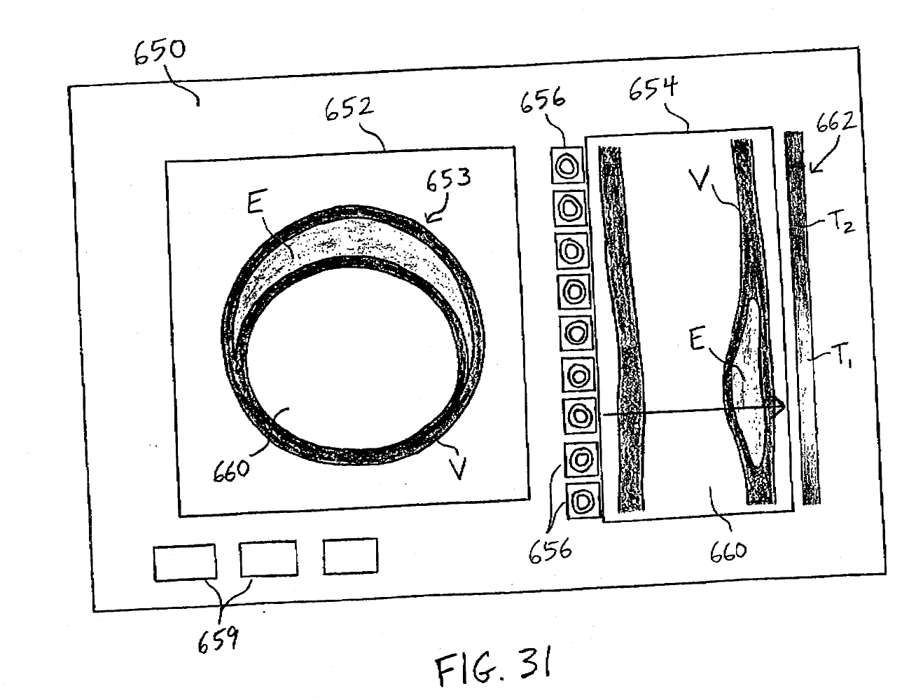

In operation, catheter 602 is percutanously advanced into a patient's vessel over guidewire 610. Catheter 602 may comprise guidewire lumen 609, which spans the length of catheter 602, or alternatively may comprise a rapid exchange side port, e.g., as shown in FIG. 26. Catheter 602 is positioned at a desired location within vessel V, and an IVUS cross-sectional image may be provided, as shown in FIG. 29A. The cross-sectional IVUS image may provide a physician with a first indication of of the character of plaque P within vessel V, e.g., as indicated by echolucent zones characteristic of lipid pools and vulnerable plaque, or highly reflective zones indicative of calcium.

Advantageously, in accordance with principles of the present invention, infrared element 603 then is used in conjunction with imaging element 608 to provide a secondary confirmation and/or characterization of plaque P. If a physician suspects the presence of vulnerable plaque P from the IVUS image, then catheter 602 may be rotated so that side port 607 faces vulnerable plaque P to direct light onto the vulnerable plaque, as shown in FIG. 29B. The presence of vulnerable plaque may be confirmed by analyzing the spectra detected by the detection means, based on a comparison of detected light with reference absoprtion curves for various compounds. These compounds may indicate the presence and/or progression of plaque, including vulnerable plaque, as described previously.

Referring now to FIG. 30, an alternative embodiment of the device of FIG. 28 is described for use in detecting and characterizing vulnerable plaque. Apparatus 620 is constructed in accordance with apparatus 600 of FIG. 28, with the exception that fiber optic 604 and side port 616 terminate on a lateral surface of catheter 602 at a longitudinal position that is coincident with that of ultrasound transducer 608. The circumferential orientation of discrete ultrasound elements 612 may be interrupted at regular angular intervals to expose fiber optic 604 disposed within lumen 605. Apparatus 620 then may be used to provide a cross-sectional image of a patient's vessel and characterize and/or confirm the presence of plaque, according to techniques described in FIG. 29 hereinabove.

The infrared analysis elements described hereinabove optionally may be removed, and/or separately advanced, with respect to the imaging elements of the catheters of the present invention.

Referring now to FIG. 31, preferred imaging display techniques are provided for use in conjunction with apparatus of the present invention to facilitate detection and characterization of vulnerable plaque. In FIG. 31, image display apparatus 650, for example, a monitor that may be coupled to an image-processing computer, displays cross-sectional image 652 and side-sectional or longitudinal image 654, e.g. IVUS images. Side-sectional image 654 is constructed by stacking up a plurality of cross-sectional IVUS images along an axis of interest, for example, using a pullback technique, per se known. Specifically, as a catheter is retracted within lumen 660 of vessel V, e.g., using a pullback system, discrete cross-sectional IVUS images are displayed adjacent one another to form side sectional-image 654.

A plurality of discrete cross-sectional IVUS images are displayed on image display apparatus 650 as thumbnails 656. Thumbnails 656 preferably are disposed adjacent side-sectional image 654 at locations approximately corresponding to longitudinal locations of the thumbnail images with respect to the side-sectional image. Advantageously, a physician viewing display apparatus 650 may quickly bring up full cross-sectional images 653 at any longitudinal location in vessel V simply by clicking on a desired region in side sectional image 654, or by clicking on a thumbnail 656 of interest.

For example, in FIG. 31, the image displayed appears to be eccentric and comprises echolucent zone E, which is indicative of a shallow lipid pool. A physician may click on the region of side sectional image 654 indicated by the horizontal arrow, and the corresponding cross-sectional image will be displayed as image 653. Alternatively, a physician may click on any thumbnail 656 to bring up an enlarged view of a corresponding cross-sectional image 653.

Buttons 659 may be provided on image display 650 so that a physician may perform a range of functions, including, for example, saving a cross-sectional image 653 for later reference, and switching from viewing a still image to viewing real-time images within a patient's vessel.

Additionally, temperature, palpography, or other data may be obtained from an IVUS catheter of the present invention. Techniques for concurrently displaying both imaging and thermography data are described hereinabove. Palpographic techniques are described, for example, in U.S. Pat. No. 6,165,128 to Cespedes et al., which is incorporated herein by reference.

Referring again to FIG. 31, imaging and thermography data may be correlated and coupled prior to display, for example, using position indication techniques and/or a pullback system, then displayed in, for example, an overlaid, color-coded fashion on image display 650. Scale 662, which illustratively is color-coded, may serve as a reference scale for color-coded images within display 650, or may serve as a temperature indicator at adjacent points within side-sectional image 654.

Rapid correlation of IVUS images and temperature data within vessel V is expected to simplify, expedite and increase the accuracy of vulnerable plaque identification. Additional data may also be obtained, coupled and provided in the graphical display, for example, elastography or palpography data (not shown).

While preferred illustrative embodiments of the present invention are described hereinabove, it will be apparent to those of skill in the art that various changes and modifications may be made therein without departing from the invention. For example, the specific structures of the imaging elements, thermographers, and stabilization elements of the preferred embodiments of are provided only for the sake of illustration. Contemplated imaging elements include, but are not limited to, ultrasound transducers, linear-array ultrasound transducers, phased-array ultrasound transducers, rotational ultrasound transducers, forward-looking ultrasound transducers, radial-looking ultrasound transducers, magnetic resonance imaging apparatus, angiography apparatus, optical coherence tomography apparatus, and combinations thereof. Contemplated thermographers include, but are not limited to, thermocouples, thermosensors, thermistors, thermometers, spectrography devices, infrared thermographers, fiber optic infrared thermographers, ultrasound-based thermographers, spectroscopy devices, near infrared spectroscopy devices, and combinations thereof.

Contemplated stabilization elements include, but are not limited to, balloons, stents, coated stents, covered stents, stent grafts, eluting stents, drug-eluting stents, magnetic resonance stents, anastamosis devices, ablation devices, photonic ablation devices, laser ablation devices, RF ablation devices, ultrasound ablation devices, therapeutic ultrasound transducers, sonotherapy elements, coronary bypass devices, myocardial regeneration devices, sonotherapy devices, drug delivery devices, gene therapy devices, atherectomy devices, heating devices, localized heating devices, devices for heating in a range between about 38-44 degrees Celsius, cell apoptosis-inducing apparatus, growth factors, cytokines, plaque rupture devices, secondary-substance modifiers, therapeutic agents, contrast agents, drug capsules, tissue-type tags, extreme lipid lowering agents, cholesterol acyltransferase inhibitors, matrix metalloproteinase inhibitors, statins, anti-inflammatory agents, anti-oxidants, angiotensin-converting enzyme inhibitors, radiation elements, brachytherapy elements, local drug injection elements, gene therapy elements, photodynamic therapy elements, photoangioplasty elements, cryotherapy elements, and combinations thereof. Additional imaging elements, thermographers, and optional stabilization elements will be apparent to those of skill in the art. The appended claims are intended to cover all combinations of imaging elements, thermographers, and, optionally, stabilization elements that fall within the true spirit and scope of the present invention.

Furthermore, apparatus of the present invention may optionally be provided with an embolic protection device, such as distally-located expandable basket filter 335 of FIG. 9. Alternatively, embolic protection may be achieved with a proximally-located suction device. Embolic protection may be provided in order to capture emboli and/or other material released, for example, during stabilization of vulnerable plaque. Embolic protection devices are described,. for example, in U.S. Pat. No. 6,348,062 to Hopkins et al., and U.S. Pat. No. 6,295,989 to Connors, III, both of which are incorporated herein by reference. Additional embolic protection devices, per se known, will be apparent to those of skill in the art.

What is claimed is:

1. Apparatus for characterization of plaque within a patient's vessel, the apparatus comprising:
a catheter having a longitudinal axis and a distal region;
an imaging element disposed at the distal region; and
an infrared element disposed at the distal region,
wherein images of the patient's vessel may be constructed from data obtained with the imaging element, and
wherein chemical, thermographic or emissivity analysis of the vessel may be conducted using data obtained with the infrared element.

2. The apparatus of claim 1, wherein the images of the patient's vessel facilitate plaque characterization.

3. The apparatus of claim 2, wherein the analysis conducted using data obtained with the infrared element provides confirmation of plaque characterization determined from the images.

4. The apparatus of claim 1, wherein the apparatus is adapted for characterization of vulnerable plaque.

5. The apparatus of claim 1, wherein the infrared element is configured to be disposed within or immediately adjacent a field of view of the imaging element.

6. The apparatus of claim 1 wherein the infrared imaging element comprises a plurality of fibers.

7. The apparatus of claim 1 wherein the infrared imaging element comprises a single fiber.

8. The apparatus of claim 7 further comprising a beamsplitter adapted to transmit and receive light signals on the single fiber.

9. The apparatus of claim 1 wherein the infrared element overlaps the imaging element along the longitudinal axis of the catheter to facilitate correlation of imaging and infrared data.

10. The apparatus of claim 1 further comprising a stabilization element.

11. The apparatus of claim 10 wherein the infrared element may be replaced with the stabilization element.

12. The apparatus of claim 1 further comprising a graphical user interface for simultaneously displaying imaging and infrared data obtained with the imaging element and the infrared element, respectively.

13. The apparatus of claim 1, wherein chemical analysis comprises comparison of backscattered infrared light with reference absorption curves for various compounds.

14. The apparatus of claim 13, wherein the various compounds are chosen from the group consisting of lipoproteins, high-density lipoproteins, low-density lipoproteins, 128 KD lipoprotein, Group V Secretory Phospholipase 2, lysophosphatidylcholine, C-reactive proteins, serum amyloid A, cholesterol esters, cholesterol monohydrate, and combinations thereof.

15. The apparatus of claim 1, wherein emissivity analysis comprises heating the patient's vessel, and then detecting infrared radiation emitted by the patient's vessel.

16. The apparatus of claim 1, wherein thermography analysis comprises detecting infrared radiation naturally emitted by the patient's vessel.

17. The apparatus of claim 1, wherein the imaging element is chosen from the group consisting of intravascular ultrasound elements, phased-array intravascular ultrasound elements, rotational intravascular ultrasound elements, magnetic resonance imaging elements, optical coherence tomography elements, and combinations thereof.

18. The apparatus of claim 1, wherein the infrared element comprises a light source for illuminating the patient's vessel, and at least one detector for detecting infrared light backscattered from the vessel upon illumination.

19. Apparatus for identification of vulnerable plaque, the apparatus comprising:
a catheter having a longitudinal axis and a distal region;
an imaging element; and
an infrared element disposed on the distal region, wherein the infrared element is configured to illuminate a target site and measure reflected light, and further configured to measure naturally emitted radiation from the target site.

20. A method for characterizing plaque within a patient's vessel, the method comprising:
providing a catheter having an imaging element and an infrared element, the infrared element comprising a light source and a detector;
disposing the catheter within the patient's vessel at a region of interest;
obtaining an image of the plaque with the imaging element;
illuminating the plaque with the light source;
detecting light backscattered from the plaque with the detector;
analyzing the detected light; and
characterizing the plaque based on a comparison of the image of the plaque with the analysis of the detected, backscattered light.
